(19) United States
(12) Patent Application Publication
(10) Pub. No.: US 2024/0400972 A1
(43) Pub. Date: Dec. 5, 2024

(54) Antifouling Composition and Process for Production Thereof

(71) Applicants: Gonçalo Costa, Porto Salvo (PT); Patrick Freire, Oeiras (PT); Romana Santos, Algés (PT); Ana Cristina Silva, Torres Vedras (PT); Inês Guinote, São Domingos de Rana (PT)

(72) Inventors: Gonçalo Costa, Porto Salvo (PT); Patrick Freire, Oeiras (PT); Romana Santos, Algés (PT); Ana Cristina Silva, Torres Vedras (PT); Inês Guinote, São Domingos de Rana (PT)

(73) Assignee: BioMimetx LDA., Cantanhede (PT)

(21) Appl. No.: 18/802,805

(22) Filed: Aug. 13, 2024

Related U.S. Application Data
(63) Continuation of application No. 18/669,009, filed on May 20, 2024.
Application No. 18/669,009 is a continuation of application No. 18/432,964, filed on Feb. 5, 2024.
Application No. 18/432,964 is a continuation of application No. 18/471,077, filed on Sep. 20, 2023.
Application No. 18/471,077 is a continuation of application No. 18/171,136, filed on Feb. 17, 2023, now abandoned.
Application No. 18/171,136 is a continuation of application No. 18/045,322, filed on Oct. 10, 2022, now abandoned.
Application No. 18/045,322 is a continuation of application No. 17/708,839, filed on Mar. 30, 2022, now abandoned.
Application No. 17/708,839 is a continuation of application No. 17/554,399, filed on Dec. 17, 2021, now abandoned.
Application No. 17/554,399 is a continuation of application No. 15/178,054, filed on Jun. 9, 2016, now abandoned.
(60) Provisional application No. 62/174,349, filed on Jun. 11, 2015.

(51) Int. Cl. C12N 1/02; A01N 63/27; C12N 1/20; C12N 9/48; C12N 9/52

(52) U.S. Cl. CPC C12N 1/02; A01N 63/27; C12N 1/20; C12N 9/485; C12N 9/52; C12Y 304/00

(57) ABSTRACT

This invention concerns a method for preparing a bacterial supernatant comprising culturing a cell of Pseudomonas environmental strain PF-11; and recovering the supernatant. This invention also concerns a method for reducing the amount of a biofilm on a surface, reducing adhesion of at least one organism to a surface, or reducing microfouling or macrofouling on a surface comprising contacting the surface with a supernatant, supernatant fraction, modified supernatant or modified supernatant fraction of Pseudomonas strain PF-11; or a composition comprising a supernatant, supernatant fraction, modified supernatant or modified supernatant fraction of Pseudomonas strain PF-11, and one or more acceptable carriers. This invention also concerns a method for killing or reducing the growth of a fungus or bacterial cell, or killing or inhibiting the development of an insect or marine copepod, comprising contacting the fungus, bacteria, insect or marine copepod with a supernatant, supernatant fraction, modified supernatant or modified supernatant fraction of a Pseudomonas strain PF-11 culture; or a composition comprising a supernatant, supernatant fraction, modified supernatant or modified supernatant fraction of a Pseudomonas strain PF-11 culture, and one or more acceptable carriers. This invention also concerns a substantially pure culture of Pseudomonas strain PF-11. This invention also concerns a culture that is enriched in Pseudomonas strain PF-11. This invention also provides a method of identifying whether a bacteria is capable of producing one or more extracellular proteases capable of digesting a high molecular weight substrate.

This application is a continuation of U.S. Application Ser. No. 18/669,009, filed May 20, 2024, which is a continuation of U.S. Application Ser. No. 18/432,964, filed Feb. 2, 2024, which is a continuation of U.S. Application Ser. No. 18/471,077, filed Sep. 20, 2023, which is a continuation of U.S. Application Ser. No. 18/171,136, filed Feb. 17, 2023, which is a continuation of U.S. Application Ser. No. 18/045,322, filed Oct. 10, 2022, which is a continuation of U.S. Application Ser. No. 17/708,839, filed Mar. 30, 2022, now abandoned, which is a continuation of U.S. Application Ser. No. 17/554,399, filed Dec. 17, 2021, now abandoned, which is a continuation U.S. Application Ser. No. 15/178,054, filed Jun. 9, 2016, now abandoned, which claims benefit of U.S. Provisional Application No. 62/174,349 filed Jun. 11, 2015, the contents of each of which are hereby incorporated by reference in their entirety.

Throughout this application, various publications are referenced, including referenced in parenthesis. Full citations for publications referenced in parenthesis may be found listed at the end of the specification immediately preceding the claims. The disclosures of all referenced publications their in entireties are hereby incorporated by reference into this application in order to more fully describe the state of the art to which this invention pertains.

### BACKGROUND OF THE INVENTION

### Antimicrobial Resistance

Treatment and prevention of bacterial infections is a major Health challenge worldwide for the next decades, due to the increasing bacterial resistance to antimicrobials (Kaplan 2004). Per year, over 25000 patients die in the EU only, from infections due to multidrug-resistant bacteria, with an overall direct cost to society of 1.5 billion € (ECDC/EMA joint technical report 2009). However, antibiotics used to treat human pathogens are also used in animals to treat diseases, promote growth and improve feed efficiency (FAO/OIE/WHO 2003). As a consequence, antibiotics from both urban and agricultural sources persist in soil and aquatic environments, exerting a strong pressure that leads to the selection of resistant bacteria.

Accordingly, the presence of bacteria with antimicrobial resistance (AMR) has been detected in animal breeding facilities and slaughterhouses, in soils and wastewater, in urban and agricultural sewage waters. Most of these resistant bacteria have been shown to transfer the resistance genes to human pathogens (Martínez 2013). Therefore, the continuous use and abuse of antibiotics contributes strongly to select environmental resistant strains, able to transfer AMR mechanisms to pathogenic strains, and leading to a breaking point where any new antibiotic introduced in therapeutic use will, in a short period, trigger a selection of mechanisms that allow bacteria to become resistant to its use.

The flow of new available antibiotics has declined, with lower numbers licensed in the 1990s and the first decade of this century (Boucher et al. 2009). Therefore, there is a gap between the burden of infections due to AMR bacteria and the development of new antibiotics to tackle the problem. This is essentially due to the difficulty of developing new efficient antibiotics, easily overcome by bacteria, the regulatory difficulties to implement new antibiotics use and the fact that antibiotics are less lucrative for the pharmaceutical industry (Livermore 2011; Payne et al. 2007). Research efforts should therefore focus on developing new antibacterial strategies with original modes of action, which are both effective and will not foster AMR mechanisms.

### Biofouling

Living organisms are able to adhere and grow on the most diversified environmental, natural and synthetic surfaces. Biofouling consists in a natural process of multi-layered surface colonization when exposed to water, triggered by the accumulation of absorbed organic material, that forms a conditioning film for bacterial or micro-algae adhesion, leading to biofilms formation (Abarzua et al., Olsen et al.).

Novel methods and compositions for reducing antimicrobial resistance and biofouling are needed.

### Fungicides

Utilization of antifungal compounds produced by microbial organisms, such as antibiotics, have been highly exploited in the development of new active compounds, with a strong focus on medicinal compounds.

Several bacteria have been identified as producing a variety of classes of compounds that are antifungal and antibiotics in nature, including enzymes, siderophores, and diverse molecules such as hydrogen cyanide or ethylene.

As parasites, fungi are common and important pathogens that not only cause serious crop loss and disease in animal and human populations but also shape the composition and structure of natural biological communities. Fungicides can be used for a wide set of applications, from health and veterinary applications, to industrial like pulp production during paper manufacture and to house keeping.

In human health fungal infections represent the invasion of tissues by one or more species of fungi. Most fungal infections occur due to human exposure to a source of fungi in the nearby environment, such as the air, soil, or bird droppings. The common diseases caused by fungal infection includes finger nail and toe nail fungus, Athlete's foot, jock itch, scalp and hair infection, ringworm, fungal sinus infection, barber's itch and others. Such diseases generally cause pain, discomfort and social embarrassment to the patients. Sometimes it may even cause permanent damage and in some cases eventually be fatal to certain patients, such as organ transplant recipients and HIV/AIDS carriers.

Plants are also constantly challenged by a wide variety of pathogenic fungi. The control of fungi is important since fungal growth on plants or on parts of plants inhibits production of foliage, fruit or seed, and the overall quality of a cultivated crop. About 25% of all fungal diseases in agricultural and horticulture are caused by powdery mildew phytopathogens. Due to the vast economic ramifications of fungal propagation in agricultural and horticultural cultivations, a broad spectrum of fungicidal and fungistatic products has been developed for general and specific applications. Such examples are the use of inorganic bicarbonate, carbonate compounds, lecithin, and lime. However, these fungicidal and fungistatic products may be harmful to the environment and may pollute areas such as ground waters. Thus, there is a need for a biological solution, which provides a way to control fungi without harming the environment while protecting the plants with a minimum of phytotoxic side effects.

### Insecticides

Mosquito-borne diseases affect every year close to 700 million people globally and are responsible for more than one million deaths. Controlling mosquito-borne diseases is an established objective in the Millennium Development Goal of the World Health Organization (WHO). In addition, in Europe, such diseases have been identified as an emerging threat by the European Centre for Disease Prevention and Control. So far, mosquito control has relayed mainly on insecticide applications, with a heavy toll on the environment and increasing insecticide resistance.

Moreover, many insects are widely regarded as pests to homeowners, to picnickers, to gardeners, and to farmers and others whose investments in agricultural products are often destroyed or diminished as a result of insect damage to field crops. Particularly in areas where the growing season is short, significant insect damage can mean the loss of all profits to growers and a dramatic decrease in crop yield. Scarce supply of particular agricultural products invariably results in higher costs to food processors and, then, to the ultimate consumers of food plants and products derived from those plants.

Novel active ingredients in insecticides or innovative strategies are of paramount importance and are being searched for, namely based on natural products isolated either from plants or bacteria and to act in two main areas: agriculture and health.

### In Agriculture

Pesticides have been a major contributor to the growth of agriculture productivity and food supply. On the other hand, it has been as well a source of concern due to the human, animal and environmental side effects. This concern had manifested itself in the form of increased government regulation of pesticides application and use, an increase of the organic food demand and an increasing health-consciousness among the people. The extensive use of synthetic organic chemicals in the past decades has led to a number of long-term environmental problems. The major problems are related with accumulation of pesticides on the environment and especially on water. These worries continue, as well as worries about the safety of pesticide users. All of these facts indicate that there is a huge scope for growth of the bio-pesticides market globally.

The damage caused by insects is one of the most important factors in the reduced productivity of any crop plant species. The total annual economic losses reach approximately US$17.7 billion. Pesticides are continuously less effective to crop protection due to the fact that it has more probability to create resistance as well as the losses created due to the environmental impact that these chemicals have such as streams, rivers or waterways contamination. All of these variables contribute for an estimation of US$1 trillion of food lost or wasted globally per year.

### For Health

Mosquitoes are vectors of several human and/or animal pathogenic agents that cause diseases such as malaria, lymphatic filariasis, and arboviroses like dengue, Zika, yellow fever, chikungunya and West Nile fever. These diseases cause high levels of mortality and/or morbidity, especially in countries of subtropical and tropical regions, where they are most prevalent, with consequent social and economic impacts. Control of mosquito borne diseases is contemplated in the Millennium Development Goals (MDG) of the World Health Organization. Further, due to factors like globalization, human migrations and climatic changes, with consequent expansion of geographical mosquito vector species and/or pathogenic agents distribution, some of these diseases are spreading and emerging, e.g. chikungunya fever in Italy in 2007, 35 dengue fever in Madeira island-Portugal in 2012, and France in 2010 or re-emerging in temperate regions, such as malaria in Greece 2010.

Furthermore, with the introduction of West Nile fever in the US in 1999 and chikungunya in the Caribbean and Brazil in 2013/14 these are now, along with dengue, the most widespread arboviral mosquito borne diseases, spanning across the eastern and western hemispheres. The public health relevance of these infections is quite high, as they cause, either febrile syndromes, severe arthralgia, meningo-encephalitis, or haemorrhagic syndromes, which may lead to significant morbidity and/or mortality.

Vector control remains a fundamental tool in controlling vector-borne diseases. Although strategies Integrated Vector Control are recommended, vector control has relayed mainly on insecticide applications. Due to environmental costs and to insecticide resistance development (consequence of intensive and/or interrupted applications, these frequently due to very high, and not affordable, costs), new insecticides/formulations/strategies are being searched, namely biological ones, based on plant or bacterial products.

### Bio-Pesticides

The use of metabolites from bacterial origin in pest management has been growing, in agriculture and in the control of human/animal diseases vectors, as they are not so prone to induce resistance development, are generally more biodegradable and environmentally friendly than conventional pesticides.

The major bio-pesticide currently in use as bio-insecticide comes from the bacteria Bacillus thuringiensis (Bt), representing around 2% of the total insecticidal market. In mosquito vector control, the major bio-pesticides in use have been B.t. israelensis (Bti) and Lysinbacillus sphaericus (=Bacillus sphaericus) (Ls). Bti and Ls present a high specificity against larval stages of mosquitoes and kill the insect by disruption of the midgut tissue followed by septicemia caused probably not only by their exclusive action but also by other bacterial species. Upon sporulation, Bti and Ls produce crystal inclusions that are formed by a variety of insecticidal proteins called Cry or Cyt toxins. These toxins show a highly selective spectrum of activity, killing a narrow range of insect species. Their use has resulted in significant reduction in the use of chemical insecticides.

However, reports of possible resistance development to Bti and Ls based insecticides are appearing. On the other hand, pest control history shows that rotation of insecticides in use is advisable to avoid resistance development.

Accordingly, and combined with similar strategies adopted in agriculture pest management, new biocides, or ways to use them, are needed and have to be researched, providing a wider range of solutions to control harmful or vector insects such as mosquitoes.

### Marine Parasites

Intensive fish farming sustains substantial economic losses through the injury of fish by parasites like sea lice. Infestations with these marine copepods are difficult to avoid within net-pen based production, and is a serious issue within the aquaculture industry worldwide. The significance of the issue varies from region to region. In the sector of salmon farming industry, the control of sea lice is critical to its future sustainability. Sea lice infestation affects the growth, fecundity, and survival of their hosts because their feeding causes lesions leading to osmotic problems and secondary infections and, if untreated they can reach a level that is highly detrimental or lethal to the fish. Both wild and farmed salmonids can act as hosts to sea lice. Sea lice can move through the waters and transfer from farmed fish to wild fish and vice-versa. The possible interaction and cross-infestations of the parasite between farmed and wild fish is causing much concern. The aim for the aquaculture industry is to ensure that sea lice from fish farming facilities do not have any negative effect on the wild fish populations.

Presently, treatment of sea lice on farmed fish includes biological treatment (wrasse, cleaner fish), pharmaceutical treatment (oral treatment and bath treatments) and additive in-feed compounds. The various treatments might be combined. Anti-parasitic agents have been used to combat infestations since the early 1980's, and organophosphates were used from early 1980 until development of resistance in the mid 1990's. From that time, the synthetic pyrethroids cypermethrin, deltamethrin, and the avermectin emamectin almost completely replaced the organophosphates for treatment of sea several treatment failures with these lice. Lately, however, pyrethroids and emamectin have been reported, and reduced sensitivity has been detected. The strategies for pest management today rely on very few anti-parasitic agents.

Hydrogen peroxide is also used to remove sea lice from fish. However, the large volumes of hydrogen peroxide needed, limited therapeutic activity and toxicity for the fish, do not make this an ideal method. Hydrogen peroxide does not kill sea lice, so the parasite might re-attack the fish.

Even though numerous treatments are available, no reliable methods have been established yet. In addition, reduced sensitivity of sea lice to treatments has been recorded in areas subjected to frequent use. Cross-resistance may also occur between related compounds. Where there is evidence of resistance to a particular treatment, care should be taken to avoid use of related compounds. The potential for resistance can be reduced by following correct treatment procedures, administering the full-recommended dose and by alternating use of different treatment methods where possible. Hence, to avoid resistance development it is necessary that several different groups of effective compounds are available for treatment of sea lice infestations. Consequently, there is a long felt need for improved means of controlling sea lice in fish, which are effective in combating sea lice infestations and safe for the fish, consumer and the environment. A seemingly obvious approach to identifying compounds useful in management of sea lice infestations would be to focus on known pesticides, such as insecticides or compounds, which have previously been shown to be effective against marine parasites. Experience has shown, however, that even effectiveness of a particular compound against other aquatic parasites is not an indicator of the compound being effective against sea lice infestations. A great number of anti-parasitic agents for fish have been tested for their effect to combat sea lice infestations. Well-known examples are: praziquantel and different benzimidazoles (fenbendazole, mebendazole, albendazole, flubendazole, etc.) being anti-helmintics but without any effect on another anti-helminitcs (antinematodal sea lice. Pyrantel is thiophene) without effect on sea lice. Anti-protozoal agents such as toltrazuril and diclazuril (coccidiostats) are also without effect on sea lice. The same is true for bazitrazin having effect on intestine protozoa, but without effect on sea lice. Only a very limited number of the available pesticides have shown good efficacy against fish parasites like sea lice. These include the pyrethroids such as cypermethrin and deltamethrin. There are several factors explaining the difficulties experienced when known anti-parasitic compounds have been tested on new species, such as the large genotypic and phenotypic diversity between the various species of parasites, the large metabolic differences and the fact that the parasites occupy very different habitats and have different strategies for transmission and infection of the host.

The principle behind therapeutic chemicals for treating parasite infestations is to find the therapeutic window that allows for efficient inactivation of the parasite without affecting the host dramatically.

### SUMMARY OF THE INVENTION

This invention provides a method for preparing a bacterial supernatant comprising culturing a cell of Pseudomonas environmental strain PF-11; and recovering the supernatant.

This invention also provides a method for reducing the amount of a biofilm on a surface, comprising contacting the surface with a supernatant, supernatant fraction, modified supernatant or modified supernatant fraction of Pseudomonas strain PF-11; or a composition comprising a supernatant, supernatant fraction, modified supernatant or modified supernatant fraction of Pseudomonas strain PF-11, and one or more acceptable carriers.

This invention also provides a method for reducing adhesion of at least one organism to a surface, comprising contacting the surface with a supernatant, supernatant fraction, modified supernatant or modified supernatant fraction of a Pseudomonas strain PF-11 culture; or a composition comprising a supernatant, supernatant fraction, modified supernatant or modified supernatant fraction of a Pseudomonas strain PF-11 culture, and one or more acceptable carriers.

This invention also provides a method for reducing microfouling or macrofouling on a surface, comprising contacting the surface with a supernatant, supernatant fraction, modified supernatant or modified supernatant fraction of a Pseudomonas strain PF-11 culture; or a composition comprising a supernatant, supernatant fraction, modified supernatant or modified supernatant fraction of a Pseudomonas strain PF-11 culture, and one or more acceptable carriers.

This invention also provides a method for killing or reducing the growth of a fungus or bacterial cell, or killing or inhibiting the development of an insect or marine copepod comprising contacting the fungus, bacteria, insect or marine copepod with a supernatant, supernatant fraction, modified supernatant or modified supernatant fraction of a Pseudomonas strain PF-11 culture; or a composition comprising a supernatant, supernatant fraction, modified supernatant or modified supernatant fraction of a Pseudomonas strain PF-11 culture, and one or more acceptable carriers.

This invention also provides a substantially pure culture of Pseudomonas strain PF-11.

This invention also provides a method of identifying whether a bacteria is capable of producing one or more extracellular proteases capable of digesting a high molecular weight substrate comprising: i) placing cells of the bacteria in a growth limiting medium supplemented with the high molecular weight substrate; ii) determining whether the cells grow in the growth limiting medium supplemented with the high molecular weight substrate; and iii) identifying the bacteria as capable of producing one or more extracellular proteases capable of digesting the high molecular weight substrate if the cells are determined to grow in step ii), and identifying the bacteria as incapable of producing one or more extracellular proteases capable of digesting the high molecular weight substrate if the cells are determined to not grow in step ii).

### BRIEF DESCRIPTION OF THE DRAWINGS

FIG. 1A. Antimicrobial impact of bacterial secretomes. The potential of growth inhibition of the collected supernatants tested on non-pathogenic strain Pseudomonas aeruginosa ATCC27853,

FIG. 1B. Antimicrobial impact of bacterial secretomes. The potential of growth inhibition of the collected supernatants tested on non-pathogenic strain Escherichia coli ATCC25922

FIG. 1C. Antimicrobial impact of bacterial secretomes. The potential of growth inhibition of the collected supernatants tested on non-pathogenic strain Staphylococcus aureus NCTC8325.

FIG. 2. Antimicrobial impact of PF-11 secretome. The antimicrobial activity of the PF-11 secretome was tested on the reference strains as in FIG. 1 and enlarged to Pseudomonas putida reference strain KT2440 and other P. putida environmental isolates.

FIG. 3A. Antimicrobial activity of PF-11 secretome fractions. The impact of secreted peptides and small molecules. The impact of this fraction tested on the growth of strains Pseudomonas aeruginosa ATCC27853, Escherichia coli ATCC25922, Staphylococcus aureus NCTC8325 and Pseudomonas putida reference strain KT2440.

FIG. 3B. Antimicrobial activity of PF-11 secretome fractions. The impact of larger molecules including proteins. The impact of this fraction tested on the growth of strains Pseudomonas aeruginosa ATCC27853, Escherichia coli ATCC25922, Staphylococcus aureus NCTC8325 and Pseudomonas putida reference strain KT2440.

FIG. 3C. Antimicrobial activity of PF-11 secretome fractions. The impact of the boiled raw secretome. The impact of this fraction tested on the growth of strains Pseudomonas aeruginosa ATCC27853, Escherichia coli ATCC25922, Staphylococcus aureus NCTC8325 and Pseudomonas putida reference strain KT2440.

FIG. 4A. HPLC patterns for Pseudomonas strains secreted peptides. Comparison between M9 medium (control), and the P. putida reference strain and PF-11 secreted peptides in Exponential phase. The reference strain has the highest proteinaceous molecular weights in high concentrations, while the strain 11 slightly overlaps in size the first contents but mostly presents peptides distributed along the several kDa.

FIG. 4B. HPLC patterns for the Pseudomonas PF-11 isolate secreted peptides. As expected after the SDS-PAGE, in comparison to Exponential Phase, the Stationary Phase secretome has significantly higher variability and levels of peptides.

FIG. 5. Determination of surface tension of the PF-11 secretome.

FIG. 6. Analysis of the degradative enzymatic activity of PF-11 secretome on crude extracts of E. coli, S. aureus and P. aeruginosa reference strains.

FIG. 7A. Growth inhibition assays of different concentrations of PF-11 secretome against Escherichia coli 0157 and methicilin-resistant S. aureus (MRSA) ATCC 33591, virulent clinical pathogenic isolates, and the non-pathogenic E. coli and S. aureus used previously.

FIG. 7B. Growth inhibition assays of different concentrations of PF-11 secretome against Escherichia coli 0157 and methicilin-resistant S. aureus (MRSA) ATCC 33591, virulent clinical pathogenic isolates, and the non-pathogenic E. coli and S. aureus used previously. Impact of the separated peptidic fraction of PF-11 secretome.

FIG. 7C. Growth inhibition assays of different concentrations of PF-11 secretome against Escherichia coli 0157 and methicilin-resistant S. aureus (MRSA) ATCC 33591, virulent clinical pathogenic isolates, and the non-pathogenic E. coli and S. aureus used previously. Impact of larger molecules fraction of PF-11 secretome.

FIG. 7D. Growth inhibition assays of different concentrations of PF-11 secretome against Escherichia coli 0157 and methicilin-resistant S. aureus (MRSA) ATCC 33591, virulent clinical pathogenic isolates, and the non-pathogenic E. coli and S. aureus used previously. Impact of boiled full secretome of PF-11.

FIG. 8A. SDS-PAGE gels with protein profile extracted from a reference strain P. putida KT2440 and seven selected environmental isolates (PF-08, PF-09, PF-11, PF-13, PF-29, PF-50 and PF-57). Intracellular global protein profiles of stationary phase bacteria grown in M9 medium. Samples loaded correspond to equivalent amounts of total protein.

FIG. 8B. SDS-PAGE gels with protein profile extracted from a reference strain P. putida KT2440 and seven selected environmental isolates (PF-08, PF-09, PF-11, PF-13, PF-29, PF-50 and PF-57). Secreted proteins recovered from the supernatant of the same strains grown in the same growth conditions, by precipitation with TCA/acetone. Loaded samples correspond to an equivalent volume of the collected supernatant, except for PF-11 that was diluted 1:8 fold to avoid overload. Lane control corresponds to non-inoculated growth medium M9.

FIG. 8C. SDS-PAGE gels with protein profile extracted from a reference strain P. putida KT2440 and seven selected environmental isolates (PF-08, PF-09, PF-11, PF-13, PF-29, PF-50 and PF-57). Profile of proteins secreted into the medium by PF-11 along the growth curve, from OD600 nm 0.1 to 1,2 (1,2 corresponds to late stationary phase). The samples applied into the gel correspond to a culture volume of 40, 30, 20, 4, and 2 ml of supernatant, respectively. M: Molecular weight marker.

FIG. 9A. Proteolytic activity of PF-11 secretome in exponential (11 EXP) and stationary phases (11 STAT), measured in μg of protease equivalent by mg of total protein in the supernatants.

FIG. 9B. Proteolytic activity of PF-11 secretome collected in stationary phase of growth against casein according to the temperature of incubation (15, 20, 25, 30, 35, 40, and 45° C.). Data is presented in relative percentage where 100% activity corresponds to 115 μg per mg of protein (see Table 1).

FIG. 9C. Enzymatic turnover evaluation: Proteolytic activity of PF-11 stationary phase secretome after overnight incubation at 37° C. (left).

FIG. 9D. Protein turnover evaluation: PF-11 secretome profile before (lane 1) and after (lane 2) overnight incubation at 37° C. Dark vertical bars in all experiments represent the standard deviation from at least three independent measurements.

FIG. 10A. 2D diagonal SDS-PAGE gels used to screen for eventual proteolysis substrates degraded by PF-11 secreted proteins. Total protein extract from the E. coli ATCC 25922 was applyed in a 1D SDS-PAGE gel, and incubated with M9 medium, as negative control and the PF-11 supernatant, for 5 h at 35° C. Second dimension was run after incubation, presenting a continuous diagonal band in the absence of proteolysis, as seen on the left gel. The arrow represents the direction of migration of the first dimension

FIG. 10B. 2D diagonal SDS-PAGE gels used to screen for eventual proteolysis substrates degraded by PF-11 secreted proteins. Same as FIG. 10A, but using a protein extract of sea urchin adhesive footprints, prepared as above described.

FIG. 11. Marine biofilms incubated with M9 medium (control), PF-11 and KT2440 cultures and supernatants (SN). Recovered petri dishes deposited in aquariums were used to test removal of attached bacteria and microalgae, after 18 and 40 hours of incubation.

FIG. 12. Sea urchin adhesive footprints incubated with M9 medium (control), PF-11 and KT2440 cultures and supernatants (SN) colored with Cristal violet. The last two slides present the absence of impact of the boiled PF-11 supernatant on the disruption of the biological glue (PF-11 SN Boiled).

FIG. 13A. Percentage growth as normalized by the inoculum of water (O), P. putida and P. aeruginosa reference strains KT2440 and NTC, respectively, and the environmental isolates PF-11 (positive control) and PF-29 (negative control). Growth in Nutrient Broth added Bovine Serum Albumin (NB+BSA);

FIG. 13B. Percentage growth as normalized by the inoculum of water (O), P. putida and P. aeruginosa reference strains KT2440 and NTC, respectively, and the environmental isolates PF-11 (positive control) and PF-29 (negative control). Growth in Nutrient Broth added of gelatin (NB+gelatin). The values represent the average of two measurements and error bars the respective standard deviation.

FIG. 14A. Percentage growth as normalized by the inoculum water (0), P. putida and P. aeruginosa reference strains KT2440 and NTC, respectively, and the environmental isolates PF-11 (positive control) and PF-29 (negative control). Growth in M9 without nitrogen sources added of Bovine Serum Albumin (M9-N+BSA). The values represent the average of two measurements and error bars the respective standard deviation.

FIG. 14B. Percentage growth as normalized by the inoculum water (0), P. putida and P. aeruginosa reference strains KT2440 and NTC, respectively, and the environmental isolates PF-11 (positive control) and PF-29 (negative control). Growth in M9 without nitrogen sources added of gelatin (M9-N+gelatin). The values represent the average of two measurements and error bars the respective standard deviation.

FIG. 14C. Percentage growth as normalized by the inoculum water (0), P. putida and P. aeruginosa reference strains KT2440 and NTC, respectively, and the environmental isolates PF-11 (positive control) and PF-29 (negative control). Growth in M9 without carbon sources added of Bovine Serum Albumin (M9-G+BSA). The values represent the average of two measurements and error bars the respective standard deviation.

FIG. 14D. Percentage growth as normalized by the inoculum water (0), P. putida and P. aeruginosa reference strains KT2440 and NTC, respectively, and the environmental isolates PF-11 (positive control) and PF-29 (negative control). Growth in M9 without carbon sources added of gelatin (M9-G+gelatin). The values represent the average of two measurements and error bars the respective standard deviation.

FIG. 14E. Percentage growth as normalized by the inoculum water (0), P. putida and P. aeruginosa reference strains KT2440 and NTC, respectively, and the environmental isolates PF-11 (positive control) and PF-29 (negative control). Growth in Pseudomonas Minimal Medium added of Bovine Serum Albumin (PMM+BSA). The values represent the average of two measurements and error bars the respective standard deviation.

FIG. 14F. Percentage growth as normalized by the inoculum water (0), P. putida and P. aeruginosa reference strains KT2440 and NTC, respectively, and the environmental isolates PF-11 (positive control) and PF-29 (negative control). Growth in Pseudomonas Minimal Medium added of gelatin (PMM+gelatin). The values represent the average of two measurements and error bars the respective standard deviation.

FIG. 15. Visual scrutiny of the selected isolates extracellular protein hydrolysis activity. Evaluation of the degradation of the superficial gelatin layer of photofilms, after being exposed to M9 complete medium grown cultures for 15 min, 8 hours, 72 hours, and 2 months.

FIG. 16A. SDS-PAGE protein profile of secreted proteins from reference strain P. putida KT2440 (1) and selected environmental isolates PF-09, PF-11, PF-29 and reference strain NTC. Secreted proteins were recovered from the supernatant by precipitation with TCA/acetone. Loaded samples correspond to an equivalent volume of the collected supernatant-On the left hand of the gel are indicated the molecular weight marker.

FIG. 16B. Extracellular protease profiles of the secreted proteins in a gelatin zymograph.

FIG. 17A. Extracellular protease profiles of the secreted proteins of Pseudomonas aeruginosa NTC 27853 reference strain environmental isolate in a gelatin zymogram after incubation of the samples with 10 mM PMSF and/or 10 mM EDTA inhibitors.

FIG. 17B. Extracellular protease profiles of the secreted proteins of Pseudomonas PF-11 environmental isolate in a gelatin zymogram after incubation of the samples with 10 mM PMSF and/or 10 mM EDTA inhibitors.

FIG. 18A. PF-11 secretome proteins grouped by taxonomic homology.

FIG. 18B. PF-11 secretome proteins grouped by molecular function.

FIG. 18C. PF-11 secretome proteins grouped by enzymatic activity.

FIG. 19. Evolution of bacterial growth curves of Cobetia marina in marine broth, following addition of PF-11 in mid-exponential phase of growth.

FIG. 20. Antimicrobial impact of PF-11 on 2 marine bacteria growth, Vibrio cholerae and Vibrio vulnificus, by broth microdilution tests, measured in % of growth determined by OD600 mm and PF-11 concentration in ppm (w/v), corresponding to mg/L.

FIG. 21. PF-11 prevention of bacterial biofilm formation, measured in % of adhered cells density determined by cristal violet coloration. PF-11 concentration is in ppm (w/v), corresponding to mg/L.

FIG. 22. Algaecide impact of PF-11 on 1 marine (Tetraselmis suecica) and 2 fresh water (Chlamydomonas reinhardtii and Pseudokirchneriella subcapitata) microalgae growth. PF-11 concentration in g/L.

FIG. 23. Viability assays of Anopheles atroparvus larvae against different concentrations of PF-11 secretome.

FIG. 24. Viability assays of different concentrations of PF-11 secretome against sea lice Copepodids.

FIG. 25. Viability assays of different concentrations of PF-11 secretome against sea lice larvae.

### DETAILED DESCRIPTION OF THE INVENTION

### Definitions

Unless otherwise defined, all technical and scientific terms used herein have the same meaning as commonly understood by a person of ordinary skill in the art to which this invention belongs.

As used herein, and unless stated otherwise or required otherwise by context, each of the following terms shall have the definition set forth below.

### General Definitions

As used herein, “about” in the context of a numerical value or range means ±10% of the numerical value or range recited or claimed, unless the context requires a more limited range.

As used herein, a “secretome” means the totality of organic molecules and inorganic elements produced and secreted by a cell. Where growth conditions are indicated, the secretome is the totality of organic molecules and inorganic elements produced and secreted by a cell under those growth conditions. It will be understood that the secretome may be recovered in cellular supernatants.

As used herein, “operably linked” refers to a juxtaposition wherein the components are configured so as to perform their usual function. For example, control sequences or promoters operably linked to a coding sequence are capable of effecting the expression of the coding sequence.

As used herein “cell of Pseudomonas strain PF-11” refers to a cell of Pseudomonas strain PF-11 or any progeny thereof. Pseudomonas strain PF-11 has been deposited under Accession No. DSM 32058 on Jun. 2, 2015 at the Leibniz-Institut DSMZ-Deutsche Sammlung von Mikroorganismen und Zellkulturen GmbH (DSMZ), Address: Inhoffenstr. 7B 38124 Braunschweig.

As used herein “substantially pure culture” of a microorganism is a culture of that microorganism in which less than about 40% (i.e., less than about: 35%; 30%; 25%; 20%; 15%; 10%; 5%; 2%; 1%; 0.5%; 0.25%; 0.1%; 0.01%; 0.001%; 0.0001%; or even less) of the total number of viable microbial (e.g., bacterial, fungal (including yeast), mycoplasmal, or protozoan) cells in the culture are viable microbial cells other than the microorganism.

As used herein “enriched in” Pseudomonas strain PF-11 cells refers to a concentration of Pseudomonas strain PF-11 cells that is higher than any concentration of Pseudomonas strain PF-11 cells found in nature.

As used herein, the term “vector” refers to a polynucleotide molecule capable of carrying and transferring another polynucleotide fragment or sequence to which it has been linked from one location (e.g., a host, a system) to another. The term includes vectors for in vivo or in vitro expression systems. As a non-limiting example, vectors can be in the form of “plasmids” which refer to circular double stranded DNA loops which are typically maintained episomally but may also be integrated into the host genome.

Aspects of the present invention relate to the production of a secretome comprising one or more extracellular proteases by culturing a cell capable of producing the one or more extracellular proteases under conditions effective to produce the one or more extracellular proteases, and recovering the a secretome comprising the one or more extracellular proteases. A preferred cell to culture is a cell of the present invention. Effective culture conditions include, but are not limited to, effective media, bioreactor, temperature, pH and oxygen conditions that permit extracellular protease production. An effective medium refers to any medium in which a cell is cultured to produce one or more extracellular proteases of the present invention. Such medium may comprise an aqueous medium having assimilable carbon, nitrogen and phosphate sources, and appropriate salts, minerals, metals and other nutrients, such as vitamins. In some embodiments, the medium is a growth limited medium which lacks or has reduced assimilable carbon, nitrogen or phosphate sources.

The present invention also provides supernatants, modified supernatants, supernatant fractions, secretomes, partially purified secretomes, and secretome fractions of cells of the invention.

### Media

Non-limiting examples of bacterial media useful in embodiments of the invention include M9 medium, M9—NH4Cl-Vit B1 medium, M9-Glucose medium, Minimal Medium for Pseudomonas, and NB, and are described below.

M9 medium: 12.8 g Na2HPO4, 3 g KH2PO4, 0.5 g NaCl, 1 g NH4Cl, 1 ml CaCl2 100 mM, 1 ml MgSO4 1M, 500 μl Vit B1 18/1 L supplemented with 20 ml glucose 20% (0).

M9—NH4Cl-Vit B1 or M9-N medium: 12.8 g Na2HPO4, 3 g KH2PO4, 0.5 g NaCl, 1 ml CaCl2 100 mM, 1 ml MgSO4 1M/1 L (0),

M9-Glucose or M9-G: 12.8 g Na2HPO4, 3 g KH2PO4, 0.5 g NaCl, 1 ml CaCl2 100 mM, 1 ml MgSO4 1M/1 L (0)

Minimal Medium for Pseudomonas: 1 g K2HPO4, 3 g KH2PO4, 5 g NaCl, 0.2 g MgSO4.7H2O, 3 mg FeCl3/1 L (Prijambada et al. 1995)

NB: 1% peptone, 0.6% beef extract, 1% NaCl (Gaby and Hadley 1957).

### Abbreviations

The following abbreviations are used herein: BSA-Bovine Serum Albumin; OD-Optical Density; MMP-Minimal Medium for Pseudomonas; M9-N-M9—NH4Cl-Vit B1; M9-G-M9-Glucose.

### Embodiments

This invention provides a method for preparing a bacterial supernatant comprising culturing a cell of Pseudomonas environmental strain PF-11; and recovering the supernatant.

In one embodiment, the cell of Pseudomonas strain PF-11 is cultured under conditions at which the cell or the cell's progeny produce at least one extracellular protease, and the supernatant comprises the at least one extracellular protease.

In some embodiments, the supernatant is recovered when the number of cultured cells is increasing at an exponential rate. In other embodiments the supernatant is recovered after the number of cultured cells has ceased to increase at an exponential rate. In other embodiments the cell is cultured in a salts medium supplemented with glucose. In other embodiments the cell is cultured in M9 medium supplemented with glucose. In other embodiments the cell is cultured in medium which lacks ammonium and thyamine. In some embodiments the cell is cultured at a temperature of about 28, 29, 30, 31, or 32° C.

In some embodiments, the method further involves dividing the supernatant or modified supernatant into a fraction of components greater than 10 kilodaltons (kDa) in size; and a fraction of components less than 10 kDa in size.

In some embodiments, the method involves separating at least one extracellular protease from one or more components of the supernatant or a fraction thereof to: reduce the salt concentration of the supernatant or a fraction thereof; reduce the water content of the supernatant a fraction thereof; or sterilize the supernatant or a fraction thereof, so as to produce a modified supernatant or a fraction thereof.

In some embodiments the method comprises adding one or more acceptable carriers to the supernatant, modified supernatant, or fraction thereof.

This invention also provides a method for reducing the amount of a biofilm on a surface, comprising contacting the surface with a supernatant, supernatant fraction, modified supernatant or modified supernatant fraction of Pseudomonas strain PF-11; or a composition comprising a supernatant, supernatant fraction, modified supernatant or modified supernatant fraction of Pseudomonas strain PF-11, and one or more acceptable carriers.

In some embodiments the biofilm is an aquatic biofilm. In some embodiments, the aquatic biofilm is: a fresh water biofilm; a fresh water biofilm which is capable of growing in a pond, lake, or river environment; a marine biofilm; or a biofilm capable of growing in a fresh or salt water aquarium.

This invention also provides a method for reducing adhesion of at least one organism to a surface, comprising contacting the surface with a supernatant, supernatant fraction, modified supernatant or modified supernatant fraction of a Pseudomonas strain PF-11 culture; or a composition comprising a supernatant, supernatant fraction, modified supernatant or modified supernatant fraction of a Pseudomonas strain PF-11 culture, and one or more acceptable carriers.

In some embodiments the organism is an algae, a sea urchin, a barnacle, or a bryozoan zooid.

This invention also provides a method for reducing microfouling or macrofouling on a surface, comprising contacting the surface with a supernatant, supernatant fraction, modified supernatant or modified supernatant fraction of a Pseudomonas strain PF-11 culture; or a composition comprising a supernatant, supernatant fraction, modified supernatant or modified supernatant fraction of a Pseudomonas strain PF-11 culture, and one or more acceptable carriers.

In some embodiments the surface is glass, fiberglass, wood, rubber, plastic, or metal. In other embodiments the surface is that of an aquarium, pool, buoy, dock, or hull of a ship or barge. In other embodiments, the surface is that of a fishing net, or other net placed in water. In other embodiments the surface is a rope. In other embodiments the surface is that of a wall or ceiling structures.

In some embodiments the composition comprising a supernatant, supernatant fraction, modified supernatant or modified supernatant fraction of a Pseudomonas strain PF-11 culture, and one or more acceptable carriers is a paint or transparent coating.

This invention also provides a method for killing or reducing the growth of a fungus, comprising contacting the fungus with a supernatant, supernatant fraction, modified supernatant or modified supernatant fraction of a Pseudomonas strain PF-11 culture; or a composition comprising a supernatant, supernatant fraction, modified supernatant or modified supernatant fraction of a Pseudomonas strain PF-11 culture, and one or more acceptable carriers.

This invention also provides a method for killing or inhibiting the development of an insect, comprising contacting the insect with a supernatant, supernatant fraction, modified supernatant or modified supernatant fraction of a Pseudomonas strain PF-11 culture; or a composition comprising a supernatant, supernatant fraction, modified supernatant or modified supernatant fraction of a Pseudomonas strain PF-11 culture, and one or more acceptable carriers.

This invention also provides a method for killing or inhibiting the development of a marine copepod, comprising contacting the marine copepod with a supernatant, supernatant fraction, modified supernatant or modified supernatant fraction of a Pseudomonas strain PF-11 culture; or a composition comprising a supernatant, supernatant fraction, modified supernatant or modified supernatant fraction of a Pseudomonas strain PF-11 culture, and one or more acceptable carriers.

This invention also provides a method for killing or reducing the growth of a bacterial cell, comprising contacting the bacterial cell with a supernatant, supernatant fraction, modified supernatant or modified supernatant fraction of a Pseudomonas strain PF-11 culture; or a composition comprising a supernatant, supernatant fraction, modified supernatant or modified supernatant fraction of a Pseudomonas strain PF-11 culture, and one or more acceptable carriers.

In other embodiments the supernatant fraction or modified supernatant fraction comprises components greater than 10 kDa in size of a Pseudomonas strain PF-11 secretome. In other embodiments the supernatant fraction or modified supernatant fraction comprises components less than 10 kDa in size of a Pseudomonas strain PF-11 secretome. In other embodiments the bacterial cell is other than a Pseudomonas spp., Pseudomonas aeruginosa, or Pseudomonas cell. In other embodiments the bacterial cell is a Staphylococcus spp., Staphylococcus aureus, or methicillin-resistant Staphylococcus aureus cell. In other embodiments the bacterial cell is an Escherichia spp., Escherichia coli, or Escherichia coli 0157 cell.

This invention also provides a substantially pure culture of Pseudomonas strain PF-11. In some embodiments the cells of the substantially pure culture have been modified to comprise an exogenous resistance gene or an exogenous polynucleotide which encodes a reporter polypeptide operably linked to a promoter. In some embodiments the cells of the substantially pure culture have been genetically modified to have increased susceptibility to an antibiotic compound compared to a corresponding cell of Pseudomonas strain PF-11. In some embodiments, the substantially pure culture is one wherein less than about 40%; 35%; 30%; 25%; 20%; 15%; 10%; 5%; 2%; 1%; 0.58; 0.25%; 0.1%; 0.01%; 0.001%; 0.0001%; or even less of the total number of viable microbial cells in the culture are viable cells other than the Pseudomonas strain PF-11 cells.

This invention also provides a culture that is enriched in Pseudomonas strain PF-11.

This invention also provides a composition comprising the cells of any one of the embodiments described herein, or a supernatant, modified supernatant, or fraction thereof, and one or more acceptable carriers.

In some embodiments, the composition: an antifouling or antimicrobial composition comprising the cell of any one of the embodiments described herein, or a supernatant, modified supernatant, or fraction thereof. In some embodiments, the composition includes one or more acceptable carriers.

This invention also provides a method of identifying whether a bacteria is capable of producing one or more extracellular proteases capable of digesting a high molecular weight substrate comprising: i) placing cells of the bacteria in a growth limiting medium supplemented with the high molecular weight substrate; ii) determining whether the cells grow in the growth limiting medium supplemented with the high molecular weight substrate; and iii) identifying the bacteria as capable of producing one or more extracellular proteases capable of digesting the high molecular weight substrate if the cells are determined to grow in step ii), and identifying the bacteria as incapable of producing one or more extracellular proteases capable of digesting the high molecular weight substrate if the cells are determined to not grow in step ii).

In one embodiment the cells are determined to grow if the number of cells in the growth limiting medium supplemented with the high molecular weight substrate increases by at least 1, 5, 10, 100, 1000, or 10,000-fold over a period of at least 0.5, 1, 3, 4, 5, or 1-24 hours.

In one embodiment the growth limiting medium is a salts medium. In another embodiment the growth limiting medium is a salts medium supplemented with glucose. In another embodiment the growth limiting medium is M9 medium supplemented with glucose. In another embodiment the growth medium lacks ammonium and thyamine. In another embodiment the growth limiting medium is maintained at a temperature of about 28, 29, 30, 31, or 32° C. In another embodiment the growth limiting medium is a liquid. In another embodiment the growth medium comprises agar.

In one embodiment high molecular weight substrate cannot pass through a cell wall or cell membrane of cells of the bacteria. In another embodiment the high molecular weight substrate must be degraded in order to be internalized and used for growth of the cell. In another embodiment the high molecular weight substrate is gelatin, casein, hemoglobin, or bovine serum albumin (BSA).

In one embodiment the cells of the bacteria of step i) are obtained from a complete medium which is diluted into the growth limiting medium comprising the high molecular weight substrate.

Any embodiment disclosed herein may be combined with any other embodiment in any manner consistent with at least one of the objects, aims, and needs disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described in connection with the embodiment may be included in at least one embodiment.

### Culturing

Methods of culturing bacterial cells will be known to persons skilled in the art and are not limited to the methods disclosed herein. For example, cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Non-limiting methods for separating an extracellular protease from one or more components of a secretome or supernatant comprising a secretome include dialysis, ultrafiltration, ultracentrifugation and chromatographic methods (including, but not limited to, ion-exchange chromatography, size-exclusion chromatography, Expanded Bed Adsorption (EBA) Chromatographic Separation, reverse-phase chromatography, Fast protein liquid chromatography, or affinity chromatography).

Non-limiting examples of methods for removing cells from culture medium to recover a supernatant comprising a secretome include centrifugation, filtration, or sedimentation. Non-limiting examples of methods for reducing the water content of a supernatant, modified supernatant, or fraction thereof include evaporation, dialysis or filtration with a low molecular weight membrane, freeze drying, spray drying and drum drying.

### Compositions of the Present Invention

Compositions of the present invention include excipients, also referred to herein as “acceptable carriers”. An excipient can be any material that a surface to be treated can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal or o-cresol, formalin and benzyl alcohol. Excipients can also be used to increase the half-life of a composition, for example; but are not limited to, polymeric controlled release vehicles, biodegradable implants, liposomes, bacteria, viruses, other cells, oils, esters, and glycols.

In some embodiments of the present invention, a composition of the invention is or is mixed with a coating. In some embodiments of the present invention, a composition of the invention is or is mixed with a paint. In one embodiment the paint is a water-based paint. In other embodiments the paint is an oil-based paint. In other embodiments the paint is a marine paint. In other embodiments the paint does not contain a solvent or diluent. The coating or paint may be applied to one or more of the surfaces disclosed herein, such as the glass of an aquarium, the lining of a pool, aquaculture nets, or the hull of a ship.

One embodiment of the present invention is a controlled release formulation that is capable of slowly releasing a composition of the present invention into an environment or on a surface. As used herein, a controlled release formulation comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. In some embodiments, controlled release formulations are biodegradable (i.e., bioerodible). Slow release compositions are particularly useful for use in moving water. The formulation is preferably released over a period of time ranging from about 1 to about 12 months. A preferred controlled release formulation of the present invention is capable of effecting a treatment preferably for at least about 1 month, more preferably for at least about 3 months, even more preferably for at least about 6 months, even more preferably for at least about 9 months, and even more preferably for at least about 12 months.

In an alternate embodiment, the composition is a dried composition. More preferably, a dried extract of a cell of the invention, such as a supernatant or a secretome of the invention. A liquid composition can be dried using any technique known in the art such as, but not limited to, freeze drying, spray drying and drum drying. The dried composition can be used as an additive to a coating or paint.

### Modifications of Cell

In an embodiment, a cell of the invention has been modified from its naturally occurring counterpart. In one embodiment, the cell is resistant, or has increased resistance, to an antibiotic compared to its naturally occurring counterpart. In another embodiment the cell is not resistant, or is less resistant, to an antibiotic compared to its naturally occurring counterpart.

Aspects of the present invention relate to the capacity to select for a cell of the invention with a specific genotypic alteration. In some embodiments a cell of the invention comprises an exogenous selectable marker which allows for selection of the cell.

One common selection strategy in recombinant DNA technology is to include a cloned gene or DNA sequence in a genetic element (plasmid, virus, transposon etc.) that has a phenotypical property which allows for the separation of host cells containing the element (transformed cells) from cells that do not. Particularly useful is a gene that provides for survival selection. Thus, a selection of cells containing the genetic element can conveniently be achieved by growing cells on a medium containing a toxic substance and on which only the transformants expressing the “resistance gene” are able to survive. In some embodiments, a cell of the invention comprises an exogenous gene that when expressed allows for selection of the cell. Non-limiting examples of exogenous genes which allow for selection of a cell of the invention include antibiotic resistance genes. Genes are also available that provide for virus resistance, heavy metal resistance, or polypeptide resistance. Those skilled in the art will know of protocols for the selection of transformed cells are based on genetic elements (e.g. cloning vectors) that express genes coding for resistance, such as antibiotic resistance in the transformed host cell (see e.g. U.S. Pat. No. 4,237,224; Ausubel, 2000). Illustrative antibiotics include penicillin tetracycline, streptomycin and sulfa drugs. In some embodiments cells of the invention comprise an exogenous resistance gene which is incorporated into their genomes.

In some embodiments, a cell of the invention expresses a reporter polypeptide. As used herein, a “reporter polypeptide” is a polypeptide that provides an identifiable signal within a cell, or which is capable of being specifically detected within a cell by any technique known in the art. Examples of reporter polypeptides include but are not limited to streptavidin, beta-galactosidase, epitope tags, fluorescent proteins, luminescent proteins and chromogenic enzymes.

Fluorescent proteins will be well known to one skilled in the art, and include but are not limited to GFP, AcGFP, EGFP, TagGFP, EBFP, EBFP2, Asurite, mCFP, mKeima-Red, Azami Green, YagYFP, YFP, Topaz, mCitrine, Kusabira Orange, mOrange, mKO, TagRFP, RFP, DsRed, DsRed2, mStrawberry, mRFP1, mCherry, and, mRaspberry. Examples of luminescent proteins include but are not limited to enzymes which may catalyze a reaction that emits light, such as luciferase. Examples of chromogenic enzymes include but are not limited to horseradish peroxidase and alkaline phosphatase.

Examples of epitope tags include but are not limited to V5-tag, Myc-tag, HA-tag, FLAG-tag, GST-tag, and His-tags. Additional examples of epitope tags are described in the following references: Huang and Honda, CED: conformational epitope database. BMC Immunology 7:7 a www.biomedcentral.com/1471-2172/7/7 #B1. Retrieved Feb. 16, 2011 (2006); and Walker and Rapley, Molecular biomethods handbook. Pg. 467 (Humana Press, 2008). These references in their entireties are hereby incorporated by reference into this application.

### Reference to Other Publications or References and to the Experimental Details

All publications and other references mentioned herein are incorporated by reference in their entirety, as if each individual publication or reference were specifically and individually indicated to be incorporated by reference. Publications and references cited herein are not admitted to be prior art.

This invention will be better understood by reference to the Experimental Details which follow, but those skilled in the art will readily appreciate that the specific experiments detailed are only illustrative of the invention as defined in the claims which follow thereafter.

### Experimental Details

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### Example 1—Isolation, Characterization and Preservation of Strain PF-11

### Environmental Sampling and Bacterial Isolation.

Soil and/or mud samples were collected in the Tagus river area around Lisbon, Portugal. The collected material (10 g) was homogenized with sterile water (50 mL). After gravitational settling of the mixture, the liquid fraction was recovered. Material suspensions (including microorganisms) were then collected by centrifugation (12000 g, 5 min). The resulting pellet was resuspended in sterilized water. Primary growth was performed in LB (Luria Bertani) medium. These cultures were diluted (10-2 to 10-9) and plated in either LA (LB+Agar) or LA with ampicillin (8 μg/mL), amoxicillin (8 μg/mL) or cefotaxime (2 μg/mL), to select resistant or reduced susceptibility presenting strains. Colonies with visible differences in size or morphology were selected. Each selected colony was subjected to successive plate passage (up to 3 times) to obtain pure cultures.

### Identification of Isolated Strains.

Detection of Gram-negative strains was performed in selective McConkey nr3 medium with cycloheximide. Biochemical characterization was performed to establish broad strain characteristics. TSI (Triple Sugar Iron), oxidase and catalase tests inferred the ability to ferment dextrose, lactose, sacarose, and sulphured compounds and to produce oxidases and/or catalases (Hajna 1945). According to the previous determinations, a commercially available phenotypic identification system was used to perform the accurate identification of the Gram-negative bacteria isolated. The API® (API 20E and API ID32 GN) test strips were used, coupled with an automated system and software (BioMérieux), providing identification with a precision ≥99,5%. Isolated strain PF-11 was identified as Pseudomonas putida with 99, 9% precision.

### Mic Determination.

MICS (Minimum Inhibitory Concentration) was determined by agar dilution and disk diffusion according to CLSI standards (CLSI, M02-A10; CLSI, M100-S21). Briefly, LA plates were prepared supplemented with serial dilutions of the antibiotic to be tested to determine the MIC. The lowest antibiotic concentration preventing growth of the strain is considered the MIC value. The strain Escherichia coli ATCC 25922 was used as control strain, as recommended. Strain PF-11 was determined to be resistant to ampicilin, amoxycillin, cefotaxime, ceftazidime, cefoxitine, aztreonam using CLSI standards and reference values of resistance breakpoints by EUCAST.

### Preservation of PF-11 Strain.

The strain was stored at −80° C. using LB medium supplemented with 20% glycerol as conservation medium, in several aliquots.

### Example 2—PF-11 Culture Growth, Compounds Production, Recovery and Characterization of Secreted Compounds

### PF-11 Growth Conditions.

Frozen bacterial aliquot is plated overnight (16-18 h), at 30° C. in LB agar medium. One colony is then used to inoculate a sterile flask containing M9 medium supplemented with glucose, grown at 30° C., at 120 r.p.m. in an orbital shaker for 16 h.

Recovery of Compounds from the Culture.

The cells are removed by centrifugation at 14.000 r.p.m., 4° C., 15 min and the supernatant collected. The supernatant is sterilized by filtration using a filtration device with 0.22 μm DURAPORE filters (Millex GP, Millipore, Ireland). Sterility is confirmed by incubating 50 μl of the supernatant at 30° C., for at least 16 h in LA plates.

### Purification of the Raw Mixture of Compounds.

The supernatant is frozen at −80° C. and dehydrated by lyophilization. It is then resuspended in water and a dialisis is performed to remove excess salt using a cutoff of 2 kDa. The dialized mixture is the re-lyophilized and kept at −80° C. as a powder.

### Identification of the Proteins Present in the Mixture.

Secretome proteins were separated by dodecyl sodium sulfate polyacrylamide gel electrophoresis (12% SDS-PAGE), in mini-gel format (7×7 cm Tetra system from Bio-Rad). Twenty micrograms of protein were used per lane. Samples were diluted 10 fold in MilliQ water and mixed with reduction buffer (62.5 mM Tris-HCl, pH 6.8, 20% (v/v) glycerol, 2% (w/v) SDS, 5% (v/v) b-mercaptoetanol). Prior to electrophoresis, samples were heated at 100° C. for 5 min. Protein bands were stained with Coomassie Brilliant Blue R-250.

Protein bands were manually excised from the gels and washed in MilliQ water and distained with 50% (v/v) acetonitrile and subsequently with 100% acetonitrile. Cysteine residues were reduced with 10 mM DTT and alkylated with 50 iodoacetamide. Gel pieces were dried by centrifugation under vacuum and rehydrated in digestion buffer containing 50 mM NH4HCO3 and 6.7 ng·μL-1 of trypsin (modified porcine trypsin, proteomics grade, Promega) at 4° C. After 30 min, the supernatant was removed and discarded and 20 μL of 50 mM NH4HCO3 were added. Digestions were allowed to proceed at 37° C. overnight. After digestion, the remaining supernatant was removed and stored at −20° C.

The resulting peptide mixtures were desalted with a ZipTip C18 (Millipore), vacuum dried and reconstituted in 0.1% FA prior to analysis. The nano-LC-MS/MS set up was as follows. Samples were injected through a Finnigan Micro AS autosampler and loaded to a NanoEase trap column Symmetry 300™, C18, 5 μm (Waters) at a flow rate of 15 μl/min using the Micro AS-Surveyor MS chromatographic system. Peptides were separated using a C18 PepMap 100, 3 μm capillary column (75 μm, 15 cm) (Dionex, LC Packings) with a 160 min run, comprising a 10 min isocratic elution at 0% B, three consecutive steps with linear gradients from 0% to 15% B in 10 min, from 15% to 60% B in 70 min, and from 60% to 100% B in 20 min, followed by isocratic elution at 100% B in 10 min (A=0.1% FA in H2O, B=0.1% FA in CH3CN). The 110 nl/min flow rate used for peptide separation was provided by an in-house splitter system. The column outlet was connected to the LC coupler of the TriVersa NanoMate, which was coupled to a 7 T LTQ-FT Ultra. The mass spectrometer was operated in a datadependent mode Up to ten of the most intense ions per scan were fragmented and detected in the linear ion trap. Ion transmission into the FTICR cell and the linear trap was automatically controlled for optimal performance of the analyzers by setting the charge capacity to 1 million counts for the survey full scan and to a 50,000 counts for the MS/MS experiments. Target ions already selected for MS/MS were dynamically excluded for 60 s. Database search was performed with Proteome Discoverer software v1.2 (Thermo) using Sequest and Mascot engines. The databases used were Swissprot and NCBInr.

Blast2GO program was used for functional analysis of identified proteins, which consists of three main steps: blast to find homologous sequences, mapping to collect GO-terms associated with blast hits and annotation to assign functional terms to query sequences from the pool of GO terms collected in the mapping. Functional assignment is based on GO database. Sequence data of identified proteins were uploaded as a multiple FASTA file for batch analysis by Blast2GO software. Blast step was performed against the public Swissprot database using blastp. Other parameters were kept at default values: e-value threshold of 1e-3 and a recovery of 20 hits per sequence. Furthermore, minimal alignment length (hsp filter) was set to 33 to avoid hits with matching regions smaller than 100 nucleotides. QBlast-NCBI was set as Blast mode. An annotation configuration with an e-value-hit-filter of 1.0E-6, Annotation CutOff of 55 and GO weight of 5 have been selected. The identified proteins were grouped in selected subgroups of GO categories (eg. molecular function) using the analysis tool of combined graph, with a sequence filter of 20 in order to obtain a compact representation of the information.

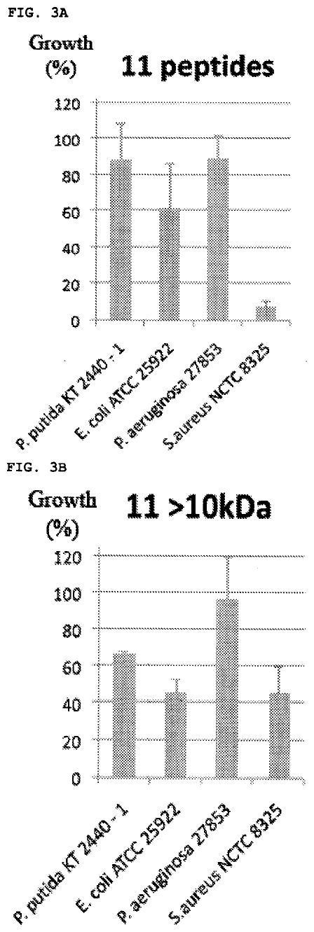

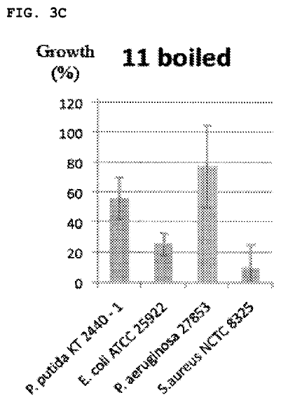

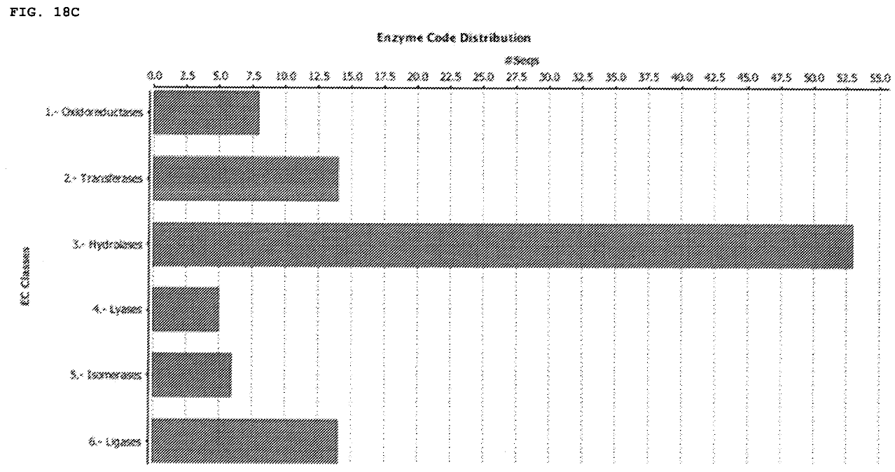

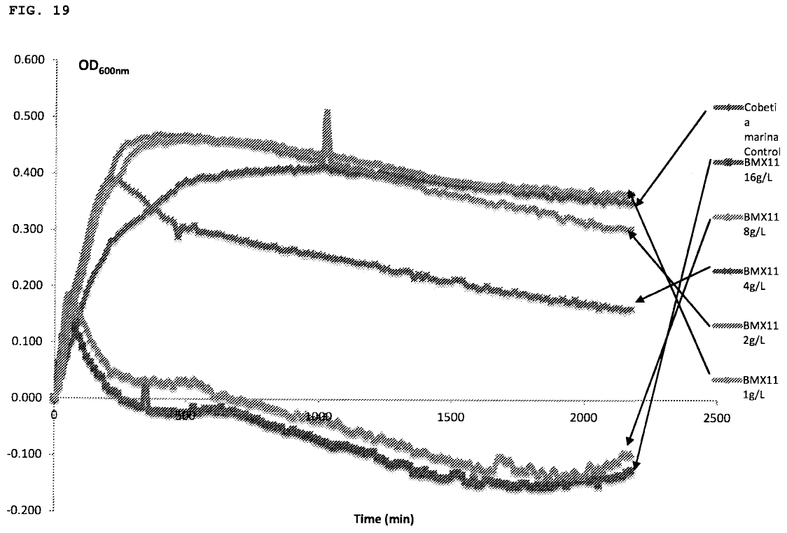

Mass spectrometry and homology search analysis revealed that PF-11 secretome contains at least 171 proteins. The functional analysis of identified proteins revealed that the secretome proteins 1) show high homology with protein from the genus Pseudomonas, and more specifically with Pseudomonas aeruginosa (FIG. 18A); 2) 36 of the secretome proteins have catalytic activity (FIG. 18B) and 3) within these, hydrolases are the most abundant enzymes in the secretome (FIG. 18C).

### Example 3—Antimicrobials from Environmental Origin

A heterogeneous collection of environmental Pseudomonas putida strains, collected in the course of previous studies through resistance to antibiotics (Meireles 2013), with strong adaptive skills, was used to screen the potential of secreted natural compounds for microbiological growth control. The contents of Meireles 2013 are hereby incorporated by reference into this application. A set of P. putida isolates from the collection was selected, based on its levels of adaptation, antibiotic resistance and general fitness (data not shown), and aiming at gathering a diversified range of strains characteristics. The secretomes (i.e. their secreted molecules) of these strains was collected and tested first for impact on the growth of three type strains of genres Escherichia coli, Staphylococcus aureus and Pseudomonas aeruginosa. One strain PF-11 revealed outstanding antimicrobial potential. Then, this initial set was enlarged to all P. putida strains from which the secretomes were collected. An initial characterization of the compounds carrying the antimicrobial properties was performed revealing the presence of peptides, enzymes and surfactants that can contribute to its impact. Finally, to test the impact of the PF-11 secretome on pathogenic strains and evaluate the potential for future application in infection treatment, the growth of clinical strains MRSA (Methicillin resistant S. aureus) and virulent E. coli 0157 were assayed. P. putida PF-11, isolated from the environment, was thus confirmed as a strong secretor of antimicrobial compounds with rich potential for future application as antibiotics.

### Materials & Methods

### Bacterial Environmental Isolates and Test Strains

Previously, a collection of 65 environmental Pseudomonas putida was isolated from soil, macroscopically selected, identified and characterized for antibiotics acquired resistance mechanisms (Meireles 2013). Soil and mud samples were collected on several locations near Tejo river in the Lisbon area, Portugal. After preliminary phenotypical analysis, the 7 P. putida strains (PF-8, PF-9, PF-11, PF-13, PF-29, PF-50, PF-57) that showed major adaptive skills (fast growth, minimal nutrient requirements) and antimicrobial multi-resistance profile were selected for screening of their secretion of antimicrobial compounds.

### Bacterial Cultures Growth Conditions

All strains used were stored at −80° C. in 5% glycerol and plated in LA (Luria broth Agar) overnight (16 hours) at 35° C. before inoculation in M9 medium. Single colonies were inoculated in liquid M9 minimal medium (50 mM Na2HPO4, 22 mM KH2PO4, 8.5 mM NaCl, 18.7 mM NH4Cl, 0.1 mM CaCl2, 1 mM MgSO4, 0.0005% Thiamine) supplemented with 0.4% glucose, at 35° C., 120 r.p.m., for 1, 2, 4, 6, or 24 hours (the latter was defined as stationary phase).

Preparation of Supernatants from Bacterial Cultures

The supernatants from bacterial cultures in M9 medium were recovered and sterile-filtered through 0.22 μm nylon filters (Millex GP, Millipore, Ireland) in a filtration device, as previously described (Roy et al.). To confirm sterility, 100 μl of each supernatant were incubated at 35° C. for at least 16 hours.

For antimicrobial experiments, filtered supernatants were used directly; for in vitro proteolytic assays, supernatants were lyophilized at −50° C. and suspended in water and 20 fold concentrated relatively to the original volume of supernatant collected.

### Cultures Crude Protein Extraction and Separation

The strains were grown overnight in LB at the indicated conditions. The cultures were then centrifuged at 10000 g for 10 min to pellet bacteria. Protein extraction buffer was added and boiled for 5 minutes to lyse the cells. The proteins were quantified in a Nanodrop device measure at 280 nm (Bio-Rad) and homogeneized to final 10 μg/10 μl. 1:1 vol of protein loading buffer with Coomassie blue Brilliant® (Sigma) and 18-mercaptoethanol was added immediately before loading the samples in the 12.5% PAA gel for SDS-PAGE.

Preparation of Supernatants from Bacterial Cultures

The selected bacterial isolates were plated for 16 hours at 35° C. and stored until extra 48 hours. Colonies were grown in Luria Bertani broth (LB) or M9 minimal medium supplemented with glucose, at 35° C., 120 r.p.m., for 1, 2, 4, 6, or 24 hours (stationary phase). After cultures had reached the aimed growth phase they were either saved for sequential use, or centrifuged at 10000 g for 15 min. Supernatants were filtered with 0.2 μm pore nylon filters (Millex GP, Millipore, Ireland) in a filtration device. In order to confirm sterility, 1 mL of each supernatant was incubated at 37° C. for 72 h. When fractioning was required the filtered supernatants were centrifuged in amicon tubes with a 10 kDa cutoff and the upper fraction was redissolved to a similar final volume in M9 medium, avoiding changes in fractions concentration or buffer composition. The heat inactivated filtered supernatants were achieved through an incubation of in 100° C. boiling water for 10 minutes, to promote proteins denaturing. Some experiments were performed with any of the immediately described fractions rested at 4° C. for 1 or 2 weeks, to “lose” proteolytic activity while maintaining the polyssacharides and surfactant properties intact and evaluate them separately to avoid unspecific reduction of molecules in fractioning procedures. At least three independent repeats were conducted for each experiment. For the HPLC analyses, the filtered supernatants (200 ml) were stored at −20° C. and lyophilized at −54° C., and finally re-suspended in 5 ml of double distilled water; the peptide fraction was separated by a 10 kDa cut-off filter so that peptides could be analyzed.

### Bacterial Proteins Degradation Studies

200 μg of total protein extracted from the E. coli type strain were mixed with the supernatants (individually) at a final concentration of 4 to 7 μg/μl and incubated at 37° C. To inhibit protease activity, 4-amidinophenylmethanesulfonyl fluoride hydrochloride was added at a final concentration of 25 μM. The contents of the reaction was mixed with loading dye (six times) and separated by SDS-PAGE. The same procedure was followed to determine the activity of the stationary phase 11 isolate secretome at different temperatures, particularly, from 15 to 45° C., every 5° C. This procedure was again used to evaluate the loss of the secretome's proteolytic activity along storage time at 4° C. A minimum of three independent repeats were conducted for each experiment.

### Detection of Proteins in Culture Supernatants

After the overnight growth of the isolates in Luria broth at the indicated conditions, cultures were centrifuged at 10000 g for 15 min to pellet bacteria, and supernatants were recovered, and sterile-filtered through a 0.2 μm filter (Millipore) as previously described (Roy et al.). Equal amounts of protein were then loaded onto adjacent wells, separated by SDS-PAGE at 12.5%.

### Separation of PF-11 Secreted Peptides by HPLC

The HPLC system consisted of a LDC, Milton Roy, Consta Metric 1 pump, and a Lichrosorb RP-18 (Merck Hibar) column (particle size of 5 μm, length—125 mm, inside diameter—4 mm). The pump pressure was 60 MPa. The injector was an automatic type (Rheotype Gilson Abimed Model 231). The detector had a fluorescence spectrophometer (Shimadzu RF 535, gamma excitation—365 mm and gamma emission—444 nm). The flow rate was 1 mL per minute, and the injection volume was 50 μL. The mobile phase was water/acetonitrile (75:25).

### Standard Solutions

AFM1, AFB1, AFB2, AFG1, and AFG2 were obtained from Sigma-Aldrich (St. Louis, MO, USA). The commercial stock solution of AFM1 was 1,000 ng/ml. The spike solution was made by diluting the stock solution 1:40 to give approximately 25 ng/ml using HPLC grade acetonitrile/water. Of the diluted stock solution, 140 μL was added to 70 mL of defatted Hipp baby milk. Calibration curve was prepared by diluting 2 μg/L of AFM1 in a 1:500 dilution. The stock solutions were stored at 4° C. when not in use.

### Results

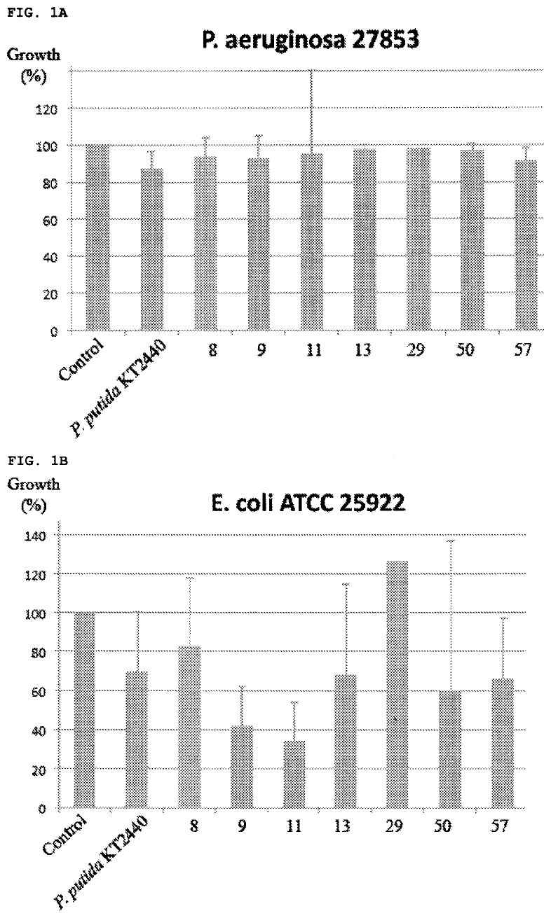

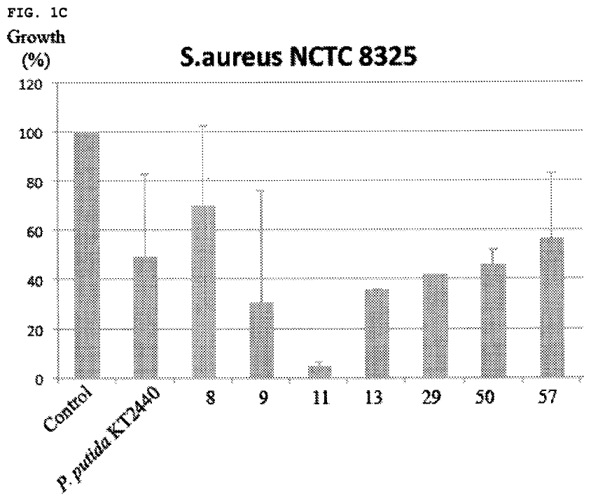

A collection of environmental Pseudomonas putida isolates gathered previously, through antibiotics selection (Meireles 2013), was used to screen the potential of natural bacterial tools for microbiological growth control. Among these strains, a few isolates revealed potential of adaptation and extracellular secretion above average (data not shown) and 7 Pseudomona environmental isolates (PF-8, PF-9, PF-11, PF-13, PF-29, PF-50, PF-57) were hence selected and further investigated. Initially, the supernatant of bacterial cultures of these selected isolates and a control strain P. putida KT2440 were collected in minimal medium and stationary phase of growth, to assess the antimicrobial impact of their secretome. The potential of growth inhibition of the collected supernatants was tested on three widely studied bacterial genres involved in human infections: Escherichia coli, Staphylococcus aureus and Pseudomonas aeruginosa. Three non-pathogenic strains (E. coli ATCC25922, S. aureus NCTC8325 and P. aeruginosa ATCC27853) were used at this stage to assess antimicrobial impact of the secretomes. The three test strains were grown in rich medium and incubated 1:1 volumes for 16 h with the 8 secretomes and a control consisting on the minimal growth medium M9 where the supernatants were recovered (FIG. 1A-C). P. aeruginosa was clearly unaffected by the compounds secreted either by the 7 isolates or the reference strain P. putida KT24240 (FIG. 1A). In contrast, both E. coli and S. aureus test strains were strongly affected by some of the P. putida secretomes. Considering only the inhibitory impact that was consistent over the different replicates, E. coli growth was inhibited by at least 40% by the secretomes of PF-9 and PF-11 (FIG. 1B). Tests with S. aureus growth inhibition showed a greater sensitivity to the secretomes, with the secretomes of strains PF-13, PF-29 and PF-50 reducing growth by more than 50%, and especially the secretome of PF-11 that impairs S. aureus growth by 90% (FIG. 1C). Considering that the secretome of strain PF-11 shows the broadest growth inhibition, both in E. coli and S. aureus, it was chosen for further analysis.

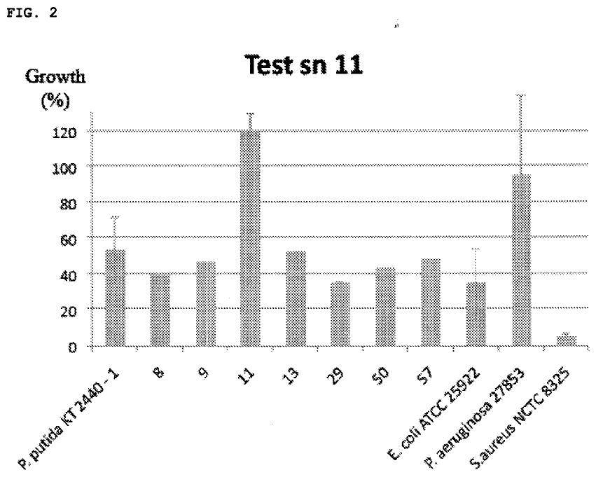

The impact on bacterial growth of the secretome of Pseudomonas PF-11 was tested as above but adding all Pseudomonas isolates and reference strain used earlier as targets. Data from FIG. 2 clearly shows that the secreted compounds recovered from the culture medium of PF-11 strain have a strong inhibitory impact, of around 50% of growth inhibition, on all P. putida strains, revealing an exceptional impact of the secreted compounds on bacteria from the same genre. In contrast, when a culture supernatant recovered from a culture of PF-11 in stationary growth phase was tested on PF-11 strain itself, growth was induced by 20% (FIG. 2), revealing the presence of growth enhancers specific to that strain, besides the growth inhibitors of other bacteria detected earlier. The presence of these growth enhancers, which are specific to PF-11, means that a substantially pure culture or a culture enriched in PF-11 will behave differently than the cells in their natural state. A substantially pure culture or a culture enriched in PF-11 will demonstrate enhanced growth compared to PF-11 cells in nature. A substantially pure culture or a culture enriched in PF-11 is therefore more useful than PF-11 cells as they exist in nature. For example, a substantially pure or enriched culture will be more useful for producing secreted compounds for antifouling, antimicrobial or other applications.

The secretome of PF-11 secreted in stationary phase of growth in minimal medium M9 was recovered and separated using 10 kDa exclusion membrane filters. Two fractions were obtained: one containing secreted peptides and small molecules, the second containing larger molecules including proteins. The impact of these fractions was tested on the growth of the strains previously used. Growth of P. aeruginosa ATCC27853 remains unaffected by any fraction of PF-11 secretome, as expectable (FIG. 3A & FIG. 3B). The growth of the two other Gram-negative strains, P. putida KT2420 and E. coli ATCC25922, was strongly impaired (50%) by the complete secretome. However, E. coli growth was only slightly reduced (25%) by the secreted peptidic fraction, while it has no significant effect on P. putida (FIG. 3A). In contrast, the fraction containing proteins and larger molecules generally retains the global antimicrobial activity observed against these two strains, even though somewhat reduced (FIG. 3B). Distinctly, S. aureus NCTC 8325 was almost completely inhibited (90%) by the complete secretome of PF-11. The data from FIG. 3A shows that the peptidic fraction of the secretome reproduces the same inhibition, indicating this fraction as the main source of anti-staphylococcal compounds. However, the proteins fraction (above 10 kDa) also presents an impact of more than 50% inhibition of growth of this strain (FIG. 3B), suggesting the presence in the secretome of several types of anti-staphylococcal molecules. To further characterize the activity of the supernatant of PF-11, its antimicrobial impact was tested after boiling to denature molecules and, among other effects, disrupt any enzymatic activity present. The data in FIG. 3C shows that the boiled secretome retains the antimicrobial impact observed previously in FIG. 2.

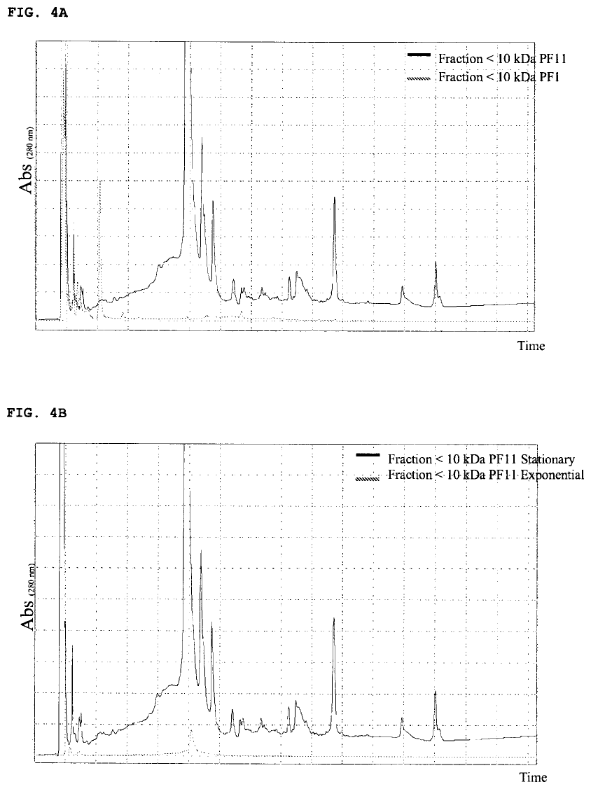

In order to characterize the secreted peptidic content (prepared as above in FIG. 3B), an assessment of the material contained in P. putida PF-11 secretome in exponential phase of growth was performed by HPLC, using P. putida KT2440 as reference strain. The profile of secreted peptides of the reference strain presented a poor elution profile with some peaks representing very small peptides visible only at the initial steps of elution. In contrast, the secretome of PF-11 showed a clear abundance of peptides, homogeneously eluted along the chromatography, and especially in much higher amounts than strain KT2440 (FIG. 4A). A more detailed HPLC analysis of this peptidic fraction of the PF-11 secretome, now comparing extracts obtained in exponential and stationary phases of growth, showed an intricate complexity of peptides secreted by this strain (FIG. 4B). Furthermore, a very strong acumulation of these small molecules is clearly detectable in stationary growth phase, revealing a good stability of the peptides and confirming strain P. putida PF-11 as an outstanding peptide secreting strain.

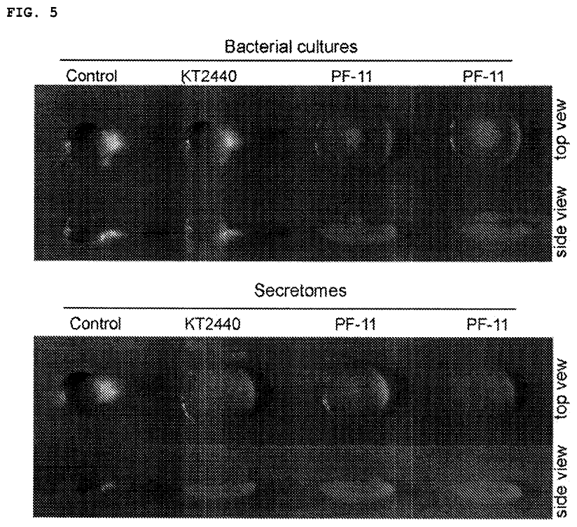

Bacterial peptides are often lipopeptides with surfactant properties. Therefore, surface tension of bacterial cultures of PF-11 in stationary phase of growth were analyzed and compared to the growth medium and strain KT2440 (FIG. 5A). Identical volumes of the solutions were dropped on a plastic surface to visualize both the diameter of the drop and its height (See Material and Methods). Both the growth medium alone and strain KT2440 showed similar features, both in diameter as in height of the deposition. In contrast, droplets of PF-11 culture revealed a very significant widening of the surface contact, clearly visible from the concomitant increase in diameter (doubled when compared to controls) and strong reduction in height. The medium with cultured PF-11 reduces strongly the contact surface tension, indicating the presence of surfactant molecules in the medium. To remove any artifactual effect from the presence of the bacterial cells, the experiment was repeated using the purified secretome of strains KT2440 and PF-11 (FIG. 5B). The data obtained previously with the bacterial cultures is confirmed with their secretomes, showing that strain PF-11 actively secretes surfactant molecules into the medium.

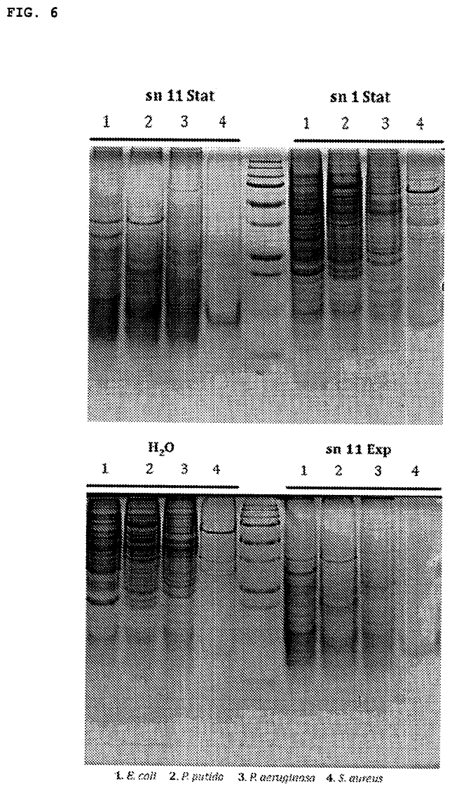

The fraction of compounds above 10 kDa in size showed significant antimicrobial impact against several of the tested target strains. Even though boiling the extracted secretome didn't alter significantly its properties, the presence of enzymes, i.e. proteins with catalytic activity, could contribute significantly to the inhibitory impact it reveals. Therefore the presence of degradative enzymes was assayed using crude protein extracts from E. coli, S. aureus and P. aeruginosa strains, incubated overnight with the secretomes of PF-11 extracted in exponential and stationary growth phase, of KT2440 and water as control (FIG. 6). Incubation with water shows the target protein pattern after one night at 37° C. without influence of external effects. The protein profiles after overnight incubation with the secretome of KT2440 causes the fainting of the top bands, corresponding to larger proteins, indicating some degradation, however at a low level. In contrast, the secretome of PF-11 strongly degrades all protein extracts, even collected in exponential phase, with a lower concentration of compounds compared to stationary phase, as seen above. This analysis reveals the presence of secreted degradative enzymes in the PF-11 secretome, in outstanding concentration and extremely efficient activity, able to degrade complex substrates such as total crude protein extracts from distinct bacteria.

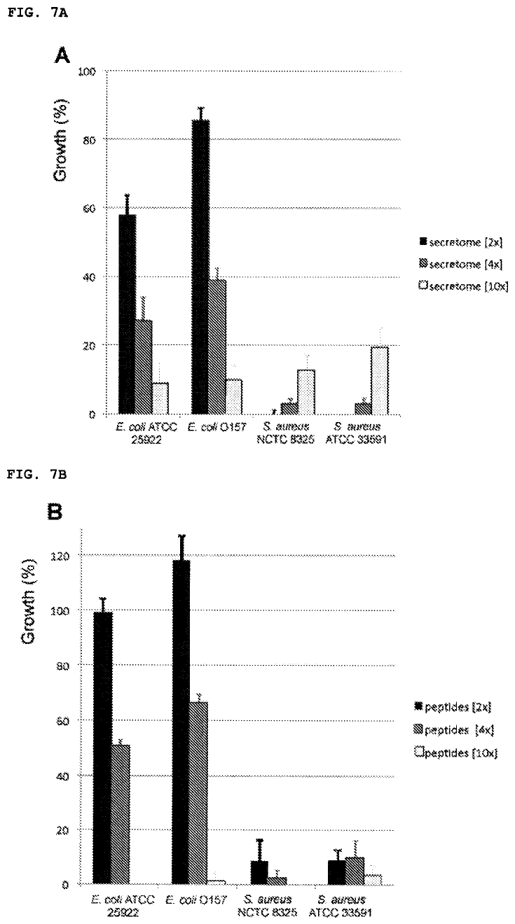

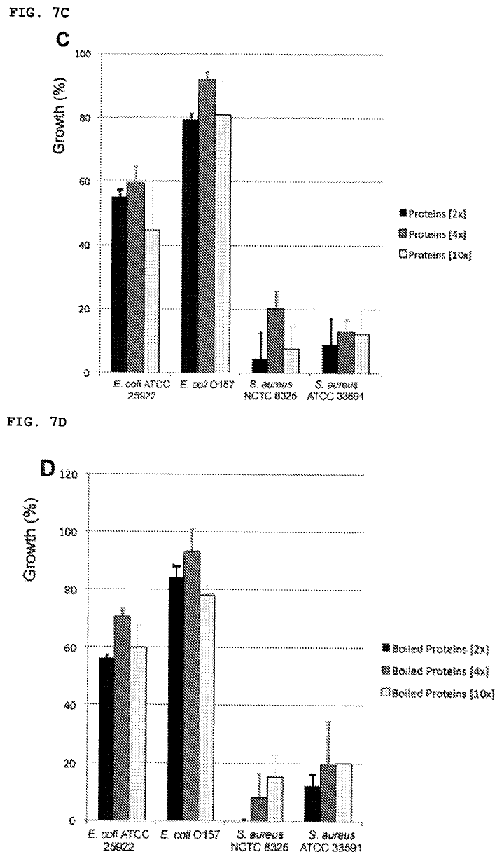

The secretome of Pseudomonas PF-11 is therefore extremely rich and complex in terms of composition and activities, with strong antimicrobial impact both on Gram-negative and -positive strains. For the potential applications of such further studies regarding compounds, it is imperative to extract and concentrate the secreted molecules, and to proceed with their characterization. Hence, the secretome was concentrated by liophilization, desalted by buffer exchange and resuspended in water. This reconstituted solution was assessed, at different concentrations (10×, 4× and 2×, with a concentration of 1× being equivalent to the concentration in the supernatant of PF-11 cultures), on the E. coli and S. aureus strains tested previously, to evaluate if the compounds retained their antimicrobial features after this purification process. Furthermore, since one aim of these studies can be to implement novel anti-infective approaches and pathogenic strains have different characteristics than “type” strains, two pathogenic strains were used to test the purified compounds secreted by PF-11. Escherichia coli 0157 and methicilin-resistant S. aureus (MRSA) ATCC 33591 are virulent clinical pathogenic isolates, generally used as reference strains in official EU controls and monitoring. First, the purified, desalted and concentrated total secretome was used in growth inhibition assays with these four strains (FIG. 7A). As before, the powerful antimicrobial activity of PF-11 secretome is clear regarding all tested strains. The S. aureus strains are totally inhibited with the 2× concentrated extract. However, with higher concentrations (4× and 10×), the inhibitory effect is reduced, but more than 80% growth inhibition is still achieved. Both E. coli strains behave similarly in the presence of the PF-11 secretome, even though E. coli 0157 is more resistant to its antimicrobial effect at lower concentrations than E. coli ATCC 25922. Regardless of this dissimilarity, at a concentration of 10×, both strains growth is inhibited in 90%. As previously, the peptidic fraction of the resuspended secretome was separated and its antimicrobial impact analyzed. Peptides show a reduced impact on E. coli strains at a concentration of 2× (FIG. 7B). However, at concentration of 4×, growth for both strains is impaired by 50% and total growth inhibition is achieved at a concentration of 10×. Regarding the peptidic anti-staphylococcal impact, an inhibition of around 90% is achieved at 2× concentration, ranging close to 100% inhibition at higher concentrations. When analyzing the antimicrobial effect of the fraction containing molecules above 10 kDA in size, the growth inhibition observed with the supernatant of PF-11 cultures on E. coli 25922 was verified (FIG. 7C), however this fraction has no significant effect on the growth of the pathogenic E. coli 0157. Surprisingly, S. aureus growth is strongly impaired for both strains, with an inhibition better than 80%, while with the supernatant only about 50% inhibition was achieved. An additional test using this same fraction, but boiled to denature molecule structures, was performed with results very similar to the non-boiled suspension (FIG. 7D).

### Example 4—Antifouling Effect of PF-11 Secretome

A heterogeneous collection of environmental strains of P. putida was collected in the course of previous studies by active selection through resistance to antibiotics (Meireles 2013). This set of strains was used to mine for extracellular secretory potential to identify and characterize proteases produced by P. putida. With comparison to P. putida KT2440, a strain isolated and studied in the context of bioremediation applications, Pseudomonas PF-11 arose as an instrumental strain with potential biotechnological relevance among all other bacteria from the collection. The selection procedure used to screen this environmental strain and the evaluation of its potential as antifouling agents secreting bacteria is described herein. A strong proteolytic impact was shown in vitro, both in the degradation of crude bacterial protein extracts and of marine biological adhesives. Furthermore, in vivo assays using either the supernatant or the whole PF-11 bacterial culture clearly prove the antifouling properties of this strain on the disruption of marine microfouling and on the degradation of biological glues produced by the sea urchin.

The strain Pseudomonas PF-11, isolated from the environment, is thus able to secrete a concentrated mixture of proteases, among other biomolecules, capable of promoting strong antifouling effects, either on microfouling or macrofouling events. Such compounds, from natural origin, are therefore potentially useful for applications in marine antifouling technologies such as additives to new coatings or protective paints.

### Materials and Methods

### Bacterial Isolates

Previously, a collection of 65 environmental Pseudomonas putida was isolated from soil, macroscopically selected, identified and characterized for acquired antibiotics resistance mechanisms (Meireles 2013). Soil and mud samples were collected on several locations near Tejo river in the Lisbon area, Portugal. After preliminary phenotypical analysis, the 7 P. putida strains that showed major adaptive skills (fast growth, minimal nutrient requirements) and antimicrobial multi-resistance profile were selected for screening of their secretory behavior. This environmental set was analyzed and compared with P. putida KT2440, a well-studied reference strain (Palleroni).

### Bacterial Cultures Growth Conditions

All strains used were stored at −80° C. in 5% glycerol and plated in LA (Luria broth Agar) overnight (16 hours) at 35° C. before M9 inoculation. Single colonies were grown in liquid M9 minimal medium (50 mM Na2HPO4, 22 mM KH2PO4, 8.5 mM NaCl, 18.7 mM NH4Cl, 0.1 mM CaCl2, 1 mM MgSO4, 0.0005% Thiamine) supplemented with 0.4% glucose (Miller et al.), at 35° C., 120 r.p.m., for 1, 2, 4, 6, or 24 hours (the latter was defined as stationary phase). After cultures reached the desired growth phase, they were either collected for direct use, or centrifuged at 10.000 g for a minimum of 15 min to separate the bulk cells in the culture medium from the secreted molecules collected from the supernatant.

### Bacterial Intracellular Protein Extraction and Separation

Bacterial cell pellets, from either exponential or stationary phase of growth, were collected by centrifugation at 10.000 g for 15 min. Bacteria were resuspended in protein extraction buffer (2% SDS, 20 mM Tris, 2 mM PMSF) (Sambrook et al.) and the suspension boiled for 5 minutes to induce cell lysis. Protein was quantified in a Nanodrop device (Thermo Fisher Scientific) by measurement at 280 nm and homogenized to final 10 μg/10 μl. 1:1 vol of protein loading buffer with Coomassie Brilliant Blue® (Sigma) (0.03%), glycerol (30%), and β-mercaptoethanol (10%) was added and boiled immediately before loading the samples in a 12.5% gel for SDS-PAGE.

Preparation of Supernatants from Bacterial Cultures

The supernatants from bacterial cultures in M9 medium were recovered and sterile-filtered through 0.22 μm nylon filters (Millex GP, Millipore, Ireland) in a filtration device, as previously described (0). To confirm sterility, 100 μl of each supernatant were incubated at 35° C. for at least 16 hours. For antifouling experiments, filtered supernatants were used directly; for in vitro proteolytic assays, supernatants were lyophilized at −50° C. and suspended in water and 20 fold concentrated relatively to the original volume of supernatant collected.

### Secreted Proteins TCA Precipitation

The sterile-filtered protein supernatants were precipitated using trichoroacetic acid (TCA) and acetone. A TCA solution at 25% in acetone at 4° C. was added to each sample at volume ratio of 1:3 (usually 8 ml TCA to precipitate 25 ml of supernatant). After homogenization the mixture was incubated on ice for 15 min and then centrifuged for 10 min, at 10.000 g, 4° C. The obtained pellet was washed twice in acetone, by suspension in 10 ml and 4 ml, respectively, followed by centrifugation in the same conditions. The dried final precipitate was suspended in 40 μl of SDS protein denaturing loading buffer (62.5 mM Tris HCl pH 6.8, 2% SDS, 5% β-mercaptoetanol, 20% glicerol, 0,01% bromophenol blue), 10 μl of which was run in a 12.5% SDS-PAGE gel. The gels were stained with Commasie Brilliant Blue (Sigma). The precipitated fractions applied to the gels correspond to 6.5 ml of the initial cell culture suspension.

### Proteolytic Assay

In order to evaluate the proteolytic content of P. putida secretome, a Fluorescent Protease Assay Kit (Pierce) was used, according to the manufacturer's instructions. Briefly, the assay involves the use of a fluorescein-labeled substrate (casein) for assessing protease activity in a sample by fluorescence resonance energy transfer (FRET). The fluorescence properties of this heavily-labeled intact protein substrate change dramatically upon digestion by proteases, which results in a measurable indication of proteolysis: the total fluorescence signal increases (as the result of a decrease of the fluorescence quenching) as the substrate is digested into smaller fluorescein-labeled fragments (homotransfer fluorescence process). The fluorescence measures were carried out with a Fluorolog-3 (Horiba Jobin Yvon) in a 0.5 cm optical path quartz cuvette, with standard fluorescein excitation/emission filters (485/538 nm). For the calibration, trypsin was the general protease chosen. Secretome samples were diluted 100 times in TBS (25 mM Tris, 0.15M NaCl, pH 7.2).

Trypsin standards and casein solution were prepared in the same buffer. All samples and standards were incubated with the substrate at room temperature for 20 min. Protein concentration was determined by the Bradford protein assay, using bovine serum albumin (BSA) as standard (Bio-Rad). The secretome of all isolated P. putidas strains were assessed according to this method, however only those that showed a total fluorescence value superior to the blanks were further treated. The estimate of protease concentration in the sample was calculated by a linear regression with the trypsin standards and then divided by the total protein amount used on the assay (μg protease/μg protein). To assess the temperature effect on protease activity the secretome samples were incubated for 20 min with the fluorescein-labeled casein at different temperatures (15 to 45° C., Δ5° C.). The highest value of μg protease activity per mg of protein obtained was considered as the maximum activity (100%) and a ratio was performed between all temperature results and this value. To assure that temperature does not influence the fluorescence signal for itself, besides the normal blanks, an extra control was made with the fluorescein-labeled casein being incubated only in buffer at each temperature studied.

### 2D-PAGE for Screening of Protease Substrates

The intracellular protein extract of a bacterial reference strain (E. coli ATCC 25922) and the sea urchins Paracentrotus lividus scrapped adhesive footprints were used as substrates for proteolysis assays as described (Nestler et al.). E. coli total proteins were extracted as above (Bacterial intracellular protein extraction and separation). Sea urchin adhesive footprints (1 mg in dry weight) were suspended in 1 mL 10% trichloroacetic acid, 0.07% B-mercaptoethanol (w/v) for 1 h at 4° C. to precipitate the proteins, then washed three times with 1 mL of cold (−20° C.) 0.07% B-mercaptoethanol in acetone (v/v), and finally vacuum dried. The obtained protein pellet was resuspended under non-reducing conditions (2% SDS, 20% glycerol, in 62.5 mM Tris-HCl pH 6.8) and the resulting solution was heated for 5 min at 95° C. Then, the sea urchin adhesive proteins were separated in a first dimension in a 12.5% polyacrylamide gels using SDS-PAGE. Following separation, lanes were excised and incubated with M9 medium, as negative control and the PF-11 strain supernatant, for 5 h at 35° C. 1D Lanes were sealed with agarose on top of 12.5% polyacrylamide gels, to run the second dimension, orthogonal to the first one. Since electrophoresis was performed under the same conditions, undigested proteins appear in a diagonal line through the gel; the products of specific proteolytic cleavages should occur below this line.

### Marine Biofilms Disruption

To evaluate ex vivo the anti-microbiological fouling potential of the PF-11 P. putida final isolate supernatant, marine biofilms were developed on Petri dishes placed inside the open-circuit tanks at 15° C. with 33% marine water, at “Vasco da Gama Aquarium” (Algés, Oeiras). These Petri dishes were gently washed with double distilled water three times to remove the excess of salts and loose organic material. The remaining strongly attached material (bacteria and microalgae) was incubated with different cell substrates and sterile supernatants to evaluate their ability to disrupt the marine microfouling from the glass matrix at room temperature for 18 and 40 hours. After the incubation period the liquid fraction was removed and the dishes gently washed with double distilled water to visualize the biofilms disruption. All tests were performed at least three times. Representative photographs are presented.

### Ex Vivo Degradation of Sea Urchin Adhesive Footprints

Sea urchins from the species Paracentrotus lividus (Lamark 1816) were collected at low tide on the west coast of Portugal (Estoril, Cascais). After collection, the animals were transported to the “Vasco da Gama Aquarium” (Algés, Oeiras) and kept in open-circuit tanks at 15° C. and 33%. Small plastic aquariums (3 L) containing sea urchins in artificial seawater (Crystal Sea, Marine Enterprises International, Baltimore, MD, USA) were used to collect the adhesive material. These aquaria were covered internally with removable glass plates to which animals were allowed to attach.

After a few hours, these glass plates, covered with hundreds of footprints, were removed, rinsed with distilled water, and either scrapped with disposable scalpels for protein extraction or used directly for removal assays (0). To evaluate the marine antifouling potential of Pseudomonas PF-11 final isolate supernatant ex vivo, the collected sea urchins footprints were incubated with the previously described cell cultures and sterile supernatants. Before incubation, the glass slides were washed under double distilled water, stained with 0.05% Crystal violet, and re-washed to visualize the adhesive footprint material (0). They were subsequently incubated with the solutions under evaluation for 18 hours, at 35° C., ˜80 r.p.m. The glass slides were finally washed and stained following the same protocol and evaluated on their ability to remove the biological adhesives from the glass substrate.

To evaluate the capacity of PF-11 secretome as an antifouling agent against invertebrates in a marine environment, the adhesive footprints secreted by sea urchin tube feet were used as a proteolytic substrate. Their strength of adhesion (force per unit of area), was estimated from 0.09 to 0.54 MPa (0 et al. 2005, Santos et al. 2006), which in comparison with other marine invertebrates (0.1-0.5 and 0.5-1 MPa for non-permanent and permanent adhesives, respectively (Smith 2006)) points sea urchin's secreted adhesive as a strong non-permanent glue.

The adhesive glue secreted by the sea urchin is able to polymerize in aqueous environments, forming an interlaced structure that contains a complex mixture of compounds, partially formed by proteins. It is therefore very stable and resistant to degradation as shown before (Santos et al. 2005, Santos et al. 2009).

Sea urchins were maintained in sea water aquariums at 15° C. and then placed on glass plates to enforce adhesion. Due to their natural behaviour, sea urchins attached and detached successively, leaving adhesive footprints on the glass, that require strong denaturing and reducing agents to be solubilized (Santos et al. 2009). A diagonal SDS-PAGE was also performed, with the first dimension consisting on the separation of proteins extracted from the adhesive footprints, followed by incubation of the gel lane with PF-11 supernatant, and finally a second dimension was run as for E. coli protein extract.

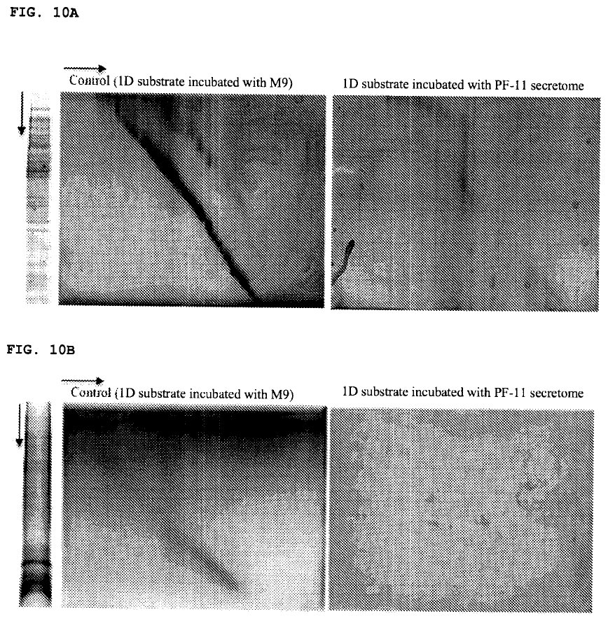

As previously, incubation with the supernatant recovered from PF-11 cell cultures ensured full degradation of the proteins. Even though this protein extract is not as complex as the total cell protein extract from E. coli, it contains adhesive proteins expected to be more challenging to degrade, however the results confirm the potent proteolytic activities of the proteins secreted by PF-11 strain and contributes furthermore to strengthen its potential as antifouling compounds secretor (FIG. 10B).

### Secretome Analysis

A collection of environmental bacterial isolates was recovered from fluvial urban shores and farm soils, using several antibiotics as a screening method to select strains with strong adaptation and survival potential. Among the bacteria gathered and identified, Pseudomonaceas emerged as an outstanding reservoir of resilience skills, probably related to their fast duplication rate, versatile adaptation to different conditions, and established resistance to stresses (Palleroni). Among that family, Pseudomonas putida was the most prevalent species and a set of 70 strains was isolated with a significant number of resistant strains to B-lactams in general (unpublished data).

To screen for potentially active biomolecules for biotechnological applications, 7 environmental isolated P. putida strains, presenting high resistance profiles to B-lactams in general, and specially to third generation cephalosporins and carbapenems, were selected, cultured in minimal mineral medium and evaluated on their secretion profiles (Meireles 2013). Initially, total cell proteins were extracted from the environmental isolates and compared to the strain P. putida KT2440, extensively studied in the context of bioremediation research (Nelson et al.).

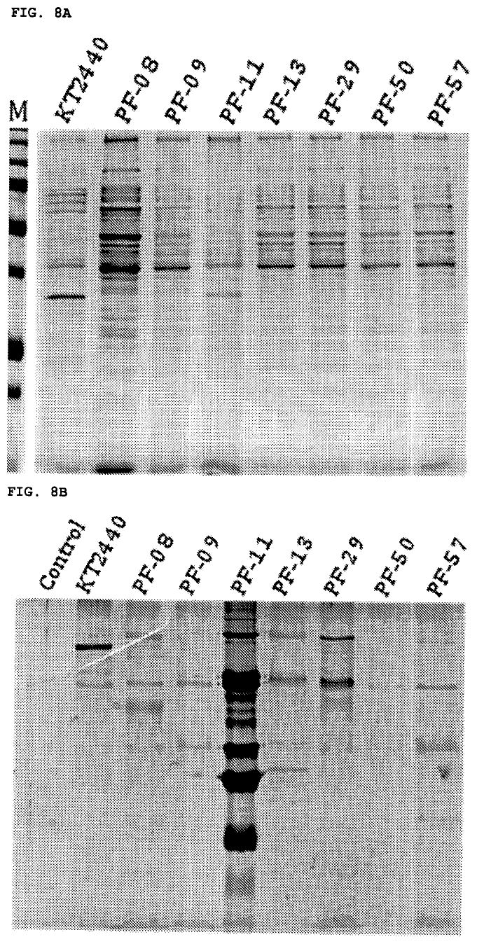

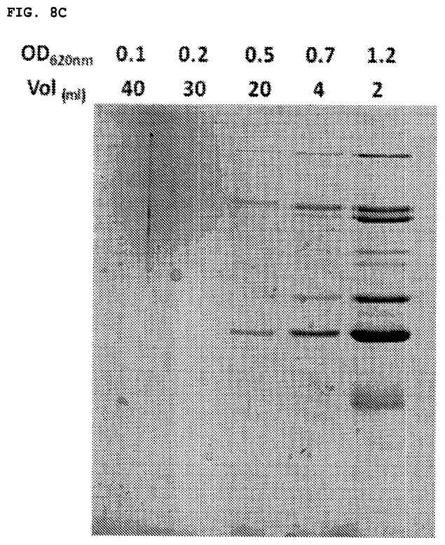

Strain KT2440 derived from P. putida mt-2, a strain isolated based on its potential as bioremediation tool and outstanding behavior, either in adaptation to adverse surroundings or resistance potential to toxic compounds (0 et al.). It is thus already a non-typical P. putida strain, in the sense that its characteristics present more versatility in terms of survival skills than most strains from the same species. Analysis by SDS-PAGE gels showed an intracellular protein distribution pattern mostly similar among the isolates (FIG. 8A) except for strain PF-11, which had a profile distribution resembling strain KT2440 (lane 4 and 1, respectively in FIG. 8A). In parallel, to estimate the secretory potential of these strains, the proteins secreted to the media were concentrated by precipitation with TCA/acetone (after removal of cells by filtration) and suspended proportionally to the initial medium volume.

The secretome profiles were similarly analyzed by SDS-PAGE (FIG. 8B). Unlike the intracellular protein fraction, the secreted proteins profiles differed amongst the isolates, not only in composition but also quantitatively. The most striking difference was observed in strain PF-11 secretory behavior. Indeed, to present data for all strains in the same gel, the recovered secretome of PF-11 strain had to be diluted 8 fold to avoid overloading. Even then, as shown in FIG. 8B, it is significantly more concentrated and complex than the secretomes of the other strains studied.

With the exception of PF-11 eight times diluted sample, the proteins loaded in this gel correspond to the same volume of culture supernatant in stationary phase of growth. Given the fact that Pseudomonas PF-11 produced such high levels of secreted proteins, revealing a strong secretory potential, its secretome was selected for further characterization. The proteins secreted by strain PF-11 were collected and visualized along the growth curve to determine variations due to growth conditions. Following the growth curve in glucose supplemented M9 minimal medium, it was possible to verify that the proteins released to the medium accumulated significantly with time due to the increase of cells in culture, as expected (FIG. 8C). The proteolytic activity of the bulk supernatants was analyzed for all the screened strains.

Filtered supernatants were lyophilized to retain enzymatic activity and suspended in water corresponding to a concentration of the raw supernatant by 20 fold. Among all samples tested, only PF-11 secretome, either collected in exponential or stationary phases of growth, was able to degrade the casein substrate for proteolytic activity determination (Table 1).

TABLE 1

| Secretome origin | μg protease/mg protein |
|---|---|
| KT2440 STAT | ND |
| PF-8 STAT | ND |
| PF-9 STAT | ND |
| PF-11 EXP | 63.15 |
| PF-11 STAT | 115.12 |
| PF-13 STAT | ND |
| PF-29 STAT | ND |
| PF-50 STAT | ND |
| PF-57 STAT | ND |

Table 1. Protease activity of total proteins secreted in stationary phase (STAT) by P. putida reference and isolate strains in M9 minimal medium. Data is also indicated for PF-11 extracellular protease in exponential phase (EXP). ND: Not Detectable.

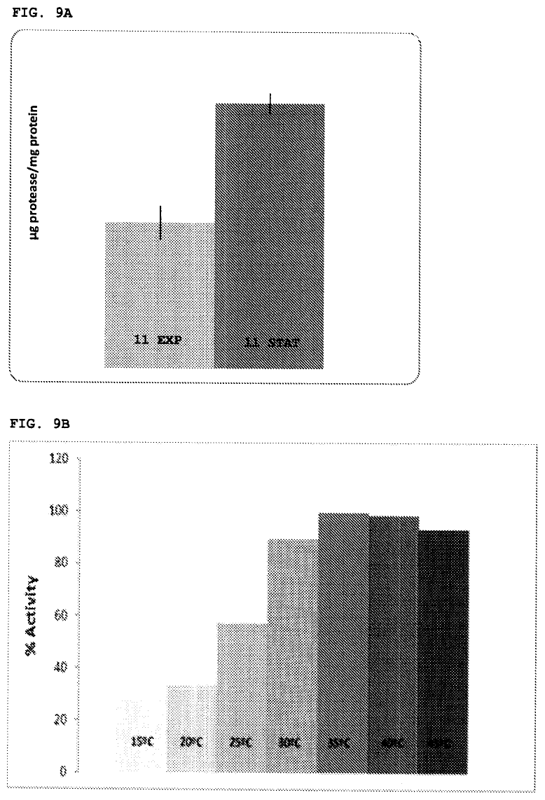

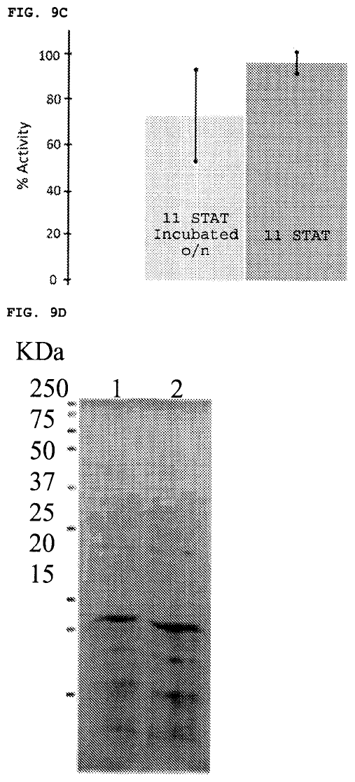

In this screening, PF-11 clearly behaves as a strain with exceptional secretory potential. An increase of proteolytic activity could be expected in secretomes collected in stationary phase, since an accumulation of global secreted proteins in the supernatant was observed along the growth curve. However, the proteolytic activity normalized by the total secreted proteins measured in stationary phase was 115 μg of protease per mg of secreted protein, corresponding to an increase of almost 2-fold when compared to exponential phase, thus also revealing enrichment in proteases among the secreted proteins along with the growth curve (Table 1; FIG. 9A).

The optimal temperature for proteolytic activity of the secreted proteases, collected in stationary phase, was measured from 15 to 45° C. and established at 35° C. (FIG. 9B). Activities were compared and plotted in percentage according to the temperatures at which proteolytic degradation of casein was tested. Since P. putida is an environmental strain, normally surviving in a range of temperatures between 15 to 30° C. in average, it could be expected that its secreted proteins would be able retain activity in a wide thermal range. As shown in FIG. 9B, at a temperature as low as 15° C. the secreted proteases still retain 30% of its proteolytic activity in comparison with its activity at the optimal temperature of 35° C.

Most bacterial proteases have optimal temperatures around 50-60° C., due to their cellular functions being exacerbated in heat shock conditions (Angilletta et al.). Extracellular bacterial proteases have also been shown to have similar features, namely in Bacillus spp, extensively used for the production and purification of proteases for industrial applications (Watanabe et al., Angilletta et al.). On the other hand, marine microorganisms usually produce cold-adapted enzymes, like a metalloproteinase produced by a marine bacterium strain isolated in China that exhibits a maximum activity at 30° C. (0 et al.).

Any industrial application for proteases will require that these enzymes possess a low turnover. To evaluate this, PF-11 secreted proteins were incubated overnight at 37° C. and the proteolytic activity was measured demonstrating that the reduction of the activity was around 25% (FIG. 9C). Moreover, the secretome protein profile after overnight incubation almost didn't change, even though some high molecular mass components were reduced probably due to autoproteolysis (FIG. 9D).

The supernatant recovered from PF-11 cultures, either in exponential or stationary phases, showed a strong proteolytic effect on casein. Even though this clearly shows a high specific proteolytic activity of this secretome, it remains a proteolytic degradation of a single substrate. In order to assess the impact of PF-11 strain secretome as a broad-range targeted protease(s) mixture, total protein was extracted from an Escherichia coli strain and used as a substrate. A diagonal SDS-PAGE gel was performed to vizualise eventual proteolysis of E. coli proteins extract by the PF-11 secretome (Nestler et al.).

Total proteins collected from E. coli ATCC 25922 were separated in one-dimensional SDS polyacrylamide gel and the gel lane was then incubated for 5 h at 37° C. with the PF-11 active supernatant. The migration of the second dimension, also separating molecules by size, clearly revealed the almost complete degradation of E. coli protein extract (FIG. 10.A). Scarce degradation fragments were still detectable, however the clear absence of a diagonal migration pattern, in contrast with the control gel, confirms the potent broad-scale proteolytic activity of the PF-11 secretome. Such results suggest that these secreted proteases can act as antifouling agents, since they were able to almost fully degrade a complex solution of proteins.

### In Vitro Effect of PF-11 Secretome

To assess in vitro the antifouling effect of the secretome of PF-11, two assays were performed. Antifouling action was tested against both microfouling and macrofouling, represented by marine bacteria, microalgae, marine biofilms and sea urchin adhesive footprints, deposited on glass Petri dishes or slides.

Material immersed in the sea become sites for the attachment of microorganisms, primarily bacteria. Many of these bacteria produce mucilaginous materials to which bits of organic detritus adhere. Microalgae may then colonize the surfaces. The bacteria and their by-products combined with the detritus and algae populations constitute what is commonly referred to as a “slime film”. This film is usually the first form of fouling to appear on a submerged surface.

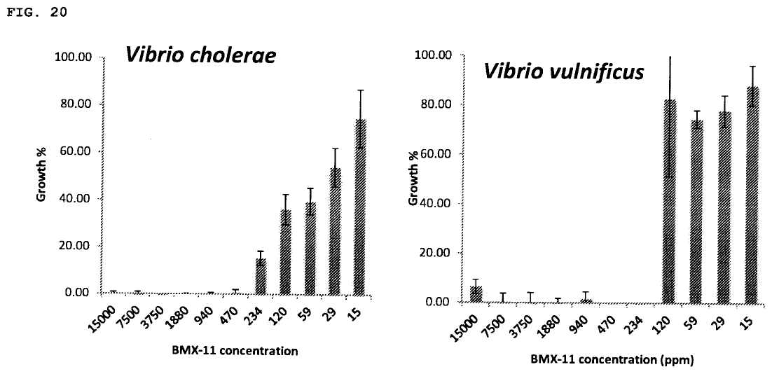

Marinobacter hydrocarbonoclasticus and Cobetia marina are strict marine bacteria consistently described as major initial colonizers of marine biofouling. These bacteria have been used regularly as indicators in marine antifouling tests, to assess the antifouling capacity of compounds directed at the initial layer of biofouling, known as microfouling or more commonly as slime. PF-11 secretome is highly efficient on the control of Cobetia marina growth. Data presented in FIG. 19 represents the growth evolution of Cobetia marina in optimal conditions in marine settings, in the presence of several concentrations of PF-11 secretome. The curves depicted clearly show a strong growth inhibition of C. marina at PF-11 secretome concentrations as low as 8 g/L and 4 g/L. PF-11 secretome also prevents the growth of several other marine bacteria in growth inhibition tests, such as Vibrio spp, commonly associated to marine biofouling, at concentrations as low as 470 or 234 ppm (w/v) (FIG. 20). Besides these strains, PF-11 secretome exerts effective antibacterial activity against bacteria, both Gram-negative and Gram-positive, namely Escherichia coli, Vibrio alginolyticus, Vibrio parahaemolyticus, Vibrio cholerae, Vibrio vulnificus, Cobetia marina, Marinobacter hydrocarbonoclasticus, Staphylococcus aureus, Enterococcus faecalis, among others.

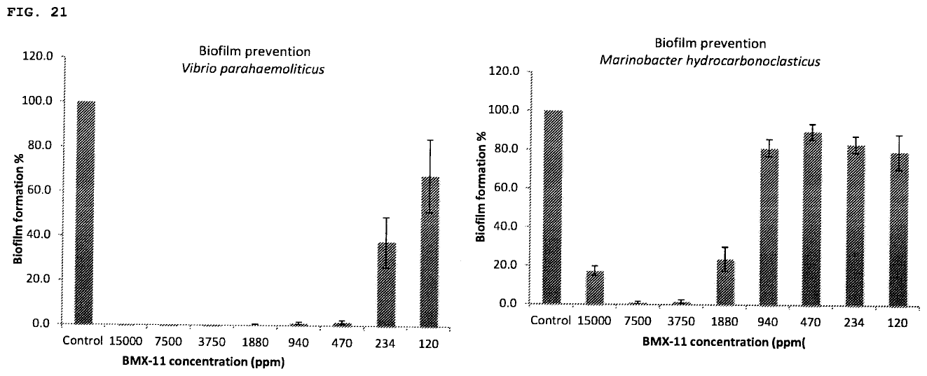

PF-11 secretome is therefore highly effective in the growth control of bacterial organisms in general. More specifically, PF-11 secretome has an antifouling capacity against the bacteria that form the initial layer of marine biofouling. Besides controlling the growth of the bacteria per se, PF-11 secretome is also efficient in preventing the formation of marine bacterial biofilm, the actual base formation of slime. Data presented in FIG. 21 shows the impact of PF-11 on the prevention of Vibrio parahaemolyticus and Marinobacter hydrocarbonoclasticus biofilms, achieved at concentrations of PF-11 secretome in the range of 500-2000 ppm.

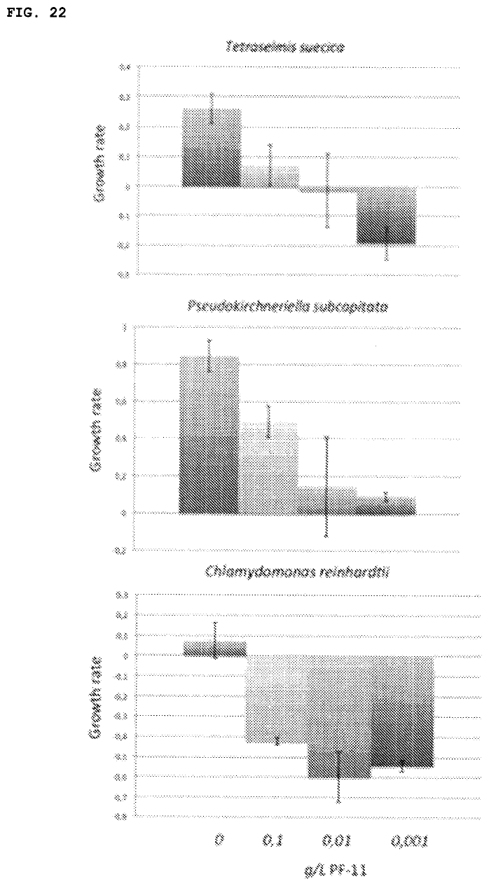

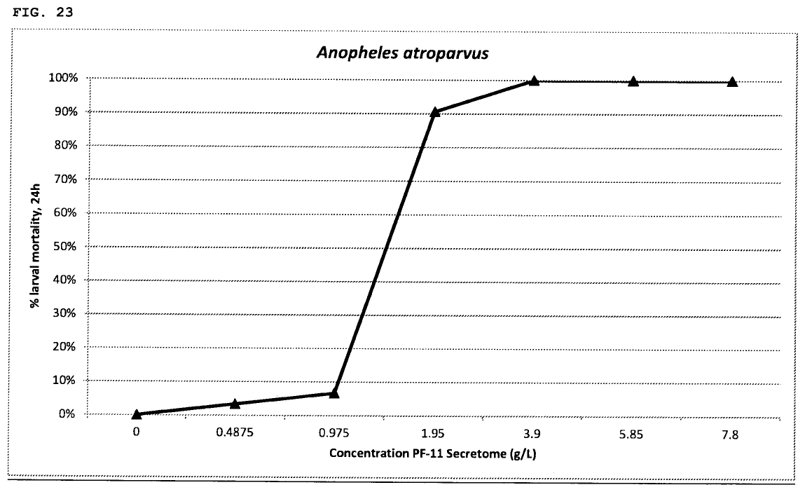

Along with marine bacteria, PF-11 secretome can also exerts effective anti-microalgae activity, affecting the growth of several micro algae species like Chlamydomonas reinhardtii, Pseudokirchneriella subcapitata and Tetraselmis suecica as can be observed in FIG. 22. The most sensitive specie tested was Chlamydomonas reinhardtii and the most resistant one was the Pseudokirchneriella subcapitata.

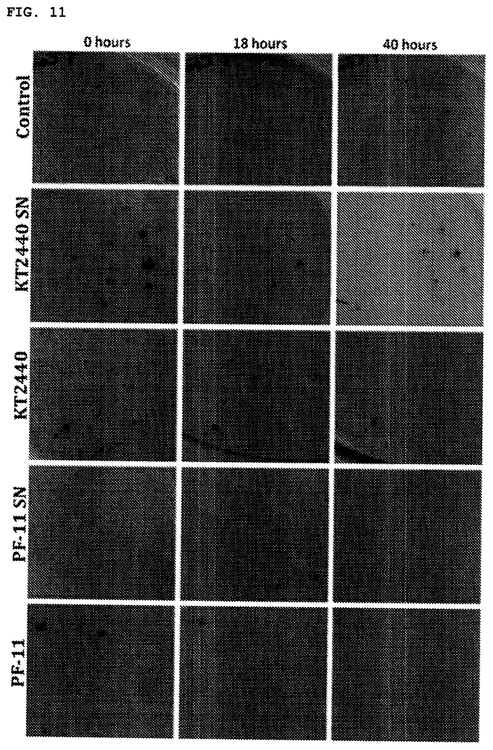

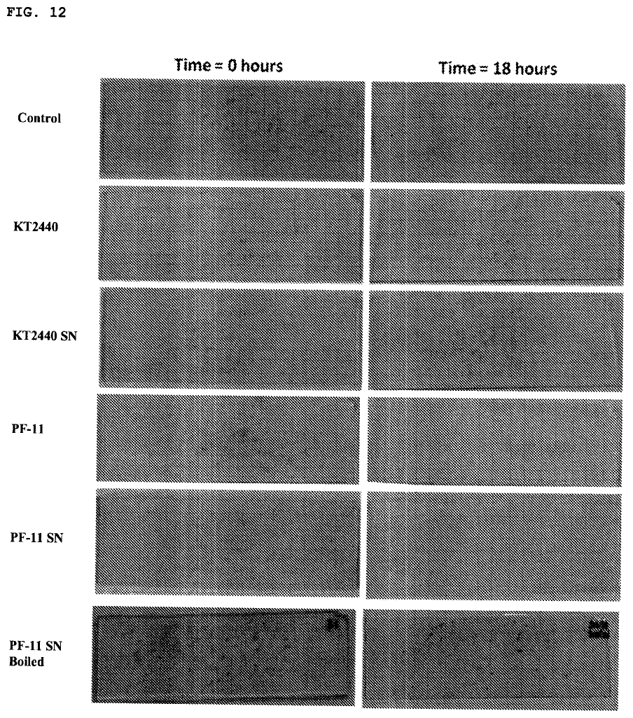

Beyond preventing the formation of single bacterial biofilms and preventing the growth of single microalgae, as shown above, PF-11 proved to effectively disrupt already formed mixed marine biofilms, composed essentially of marine bacteria algae and microalgae collected in sterilized glass Petri dishes immersed for 8 days in large aquariums with re-circulating seawater and a stabilized marine mesocosm. The resulting fouled dishes were washed to remove unattached material though retaining microfouling material composed mainly by marine bacteria and microalgae. Biofilms were then incubated with PF-11 supernatant and the culture itself, plus respective controls, and their impact assessed after 18 and 40 hours of incubation at room temperature. Poorly bound material that resisted initial washing was promptly removed (<18 hours) when incubated with either of the fractions assayed. However, the more tightly attached marine biofilm formations could only be removed by addition of the supernatant or cell culture of PF-11 (>18 hours) (FIG. 11). As for invertebrates fouling, P. putida PF-11 secretome was tested in the removal of sea urchins adhesive footprints on glass. Both cultures and supernatants recovered from the growth of PF-11 strain were able to completely disrupt and remove the adhesive footprints left by sea urchins on the glass slides (FIG. 12). In contrast, none of the supernatants or cultures used as controls, either sterile medium or material issued from distinct bacteria (including environmental isolated P. putida or KT2440 strains), exhibited any antifouling action. Since the enzymatic activity of proteins, such as proteases, is dependent on the maintenance of their native tri-dimensional structure, a supplementary assay was conducted in parallel using boiled supernatant (15 min at 100° C.) in order to assess the relevance of enzymatic activities in the observed antifouling action of PF-11 secretome. (FIG. 12) This experiment showed that native proteins, maintaining their enzymatic activity, are required to remove sea urchin secreted adhesives. Hydrolytic enzymes, such as the previously detected proteases, are thus essential to confer antifouling capacities to the compounds secreted by Pseudomonas PF-11. This environmental isolate secretes therefore a mix of highly valuable compounds, with enzymatic activities, that can strongly degrade proteins, disrupt bacterial biofilm formation and remove marine bioadhesives.

### Example 5—Enzymatic Activity of the PF-11 Secretome

The purpose of the work here described was to evaluate the proteolytic activity of secreted enzymes from PF-11 and several others isolated strains. We established a method based on the idea that only after some degree of proteolysis of high molecular weight substrates added to the protein or energy sources depleted media would the strains be able to internalize and use them for growth.

### Materials and Methods

### Bacterial Isolates

Prior to this study, a collection of 71 environmental strains was isolated, macroscopically selected, identified and characterized for acquired antibiotics resistance mechanisms (Meireles 2013). Out of the final isolates selection, 92% were Pseudomonas putida. One of them, PF-11 revealed to secrete proteases at high level. Aiming at finding more extracellular bacterial proteases potent producers, the whole collection was submitted to the screening procedures here described.

### Media and Supplements Tested

Different media referenced in the literature for this genre were evaluated, associated with different proteinaceous substrates commonly used in medium formulations or related to related screenings. The media used were: M9-12.8 g Na2HPO4, 3 g KH2PO4, 0.5 g NaCl, 1 g NH4Cl, 1 ml CaCl2 100 mM, 1 ml MgSO4 1M, 500 μl Vit B1 1%/1 L supplemented with 20 ml glucose 20% (Miller 1972), M9—NH4Cl-Vit B1 or M9-N-12.8 g Na2HPO4, 3 g KH2PO4, 0.5 g NaCl, 1 ml CaCl2 100 mM, 1 ml MgSO4 1M/1 L (Miller 1972), M9-Glucose or M9-G-12.8 g Na2HPO4, 3 g KH2PO4, 0.5 g NaCl, 1 ml CaCl2 100 mM, 1 ml MgSO4 1M/1 L (Miller 1972), Minimal Medium for Pseudomonas or MMP-1 g K2HPO4, 3 g KH2PO4, 5 g NaCl, 0.2 g MgSO4.7H2O, 3 mg FeCl3/1 L (Prijambada, Negoro et al. 1995), and NB-1% peptone, 0.6% beef extract, 1% NaCl (Gaby and Hadley 1957), mostly regarded as a positive control medium for any supplement. The supplements tested were: BSA 18, hemoglobin 18, gelatin 1%, skim-milk 2%, caseine 18, casaminoacids 18 also regarded as a positive control for all the media tested, and H2O used as a negative control.

### Minimal Media and Supplements Test for the Extracellular Protease Detection

96 wells microtiter plates were filled with 100 μl of the medium-supplement pair intended to test. A single colony of each isolate was inoculated in Bacto-Mueller Hinton medium (Difco) and grown to reach an absorbance at 600 nm about 1 or 2. The cultures were then diluted to an OD600 nm of 0.04 according to the C1V1=C2V2 formula, in M9 medium salts only and 10 μl dispensed in each microtiter plate well. The plates were incubated still at 35° C. At the time points aimed for detection-18 h, 26 h, 72 h—the plates were strappingly stirred and read in a detector. The growth was determined by the OD of the sample compared to the blank solution. The strains used for this initial test were the negative control strain PF-29 isolate, the positive control PF-11 isolate, the reference strain P. putida KT2440, and the reference strain P. aeruginosa NTC27853.

### Minimal Media Liquid Growth Procedure

96 wells microtiter plates were filled with 100 μl of the media selected: M9-N+BSA, M9-G+BSA, M9-G+gelatin, MMP+BSA, and MMP+gelatin. A single colony of each isolate was inoculated in glucose supplemented M9 complete medium and grown for about 21 hours. The cultures were then diluted 100 fold in 1× M9 salts only; and 10 μl of such dilution) dispensed in each microtiter plate well. The plates were incubated still at 35° C. At the time points aimed for detection −21 h, and 46 h—the plates were strappingly stirred and read in a detector. The growth was determined by the OD of the sample subtracted of the blank solution. All the strains from the collection were evaluated by this test conditions-again, the PF-29 isolate was used as negative, and the PF-11 isolate as positive control.

### Photo Film Gelatin Clearance Procedure

A single colony of each selected isolate was inoculated Bacto-LB (Difco) or M9 medium (0) and grown overnight. A fragment of used photofilm was added to each tube and incubated still at RT. The films were removed from the cultures at 8 min, 12 min, 1 hour, 8 hours, 32 hours, and 2 months of incubation (time that took for all photofilms gelatin layer to be degraded, namely the non-inoculated medium control). At those time points the films were washed under a bidistilled water squirt, air dried and photographed.

Preparation of Supernatants from Bacterial Cultures

Selected bacterial isolates individual colonies were inoculated in 400 ml M9 minimal medium supplemented with glucose, at 35° C., 120 r.p.m., for 24 hours. Cultures were centrifuged at 10.000 g for 15 min to pellet the bacteria, and the supernatants filtered with 0.2 μm DURAPORE low protein binding filters (Millipore) through a filtration device, as previously described (0). The recovered filtrate was frozen at −20° C., lyophilized at −52° C., and 20 times concentrated. Protein concentration was determined by the Bradford protein assay, using bovine serum albumin (BSA) as standard (Bio-Rad).

### Proteolytic In Vitro Assays.

Fluorescent Protease Assay Kit (Pierce) was used to evaluate the proteolytic content of P. putida secretome, according to the manufacturer's instructions. Briefly, the assay involves the use of a fluorescein-labeled substrate which (casein, resembles natural substrates of most proteases) for assessing protease amount in a sample by fluorescence resonance energy transfer (FRET). The fluorescence properties of the substrate change upon digestion by proteases, results in a measurable indication of proteolysis. The fluorescence measures were carried out with a Fluorolog-3 (Horiba Jobin Yvon) in a 0.5 cm optical path quartz cuvette, with standard fluorescein excitation/emission filters (485/538 nm). For the calibration, trypsin was used as standard. Secretome samples were diluted 100 times in TBS (25 mM Tris, 0.15M NaCl, pH 7.2). All samples and standards were incubated with the substrate at room temperature for 20 min. Protein concentration was determined by the Bradford protein assay, using bovine serum albumin (BSA) as standard (Bio-Rad). The quantification of protease in the sample was calculated by a linear regression with the trypsin standards and then normalized dividing the activity measured by the total protein amount used on the assay (μg protease/mg protein).

### Polyacrylamide Gel Electrophoresis

Proteins were separated by sodium dodecyl sulfate polyacrilamide gel electrophoresis (12% SDS-PAGE) as described (Lamy, et al. 2010; da Costa et al. 2011), briefly, in mini-gel format (7×7 cm Tetra system from Bio-Rad). Protein concentration was determined by the Bradford protein assay, using bovine serum albumin as standard. Samples were diluted 6 fold in reduction buffer (62.5 mM Tris-HCl, pH 6.8, 20% (v/v) glycerol, 2% (w/v) SDS, 5% (v/v) b-mercaptoetanol). Prior to electrophoresis, samples were heated at 100° C. for 5 min. Protein bands were stained with Coomassie Brilliant Blue R-250.

### Zymograms for Protease Detection

Casein and gelatin zymograms were carried out as described previously (Ołdak and Trafny 2005). Briefly, 12% SDS-polyacrylamide gels (Laemmli 1970) were co-polymerized with 1% casein or gelatin at 4° C. Non reducing loading buffer (62.5 mM Tris, 2% SDS, 10% glycerol, 0.001% bromophenol blue) was added to secretome samples prior to loading. Electrophoresis was performed at 4° C. and 100 V until the bromophenol dye reached the bottom of the gel. After electrophoresis, gels were rinsed two times in 2.5% (v/v) Triton X-100, 30 min each, for SDS removal, followed by three washes, 5 min each, in deionized water. Gels were then incubated in activation buffer (0.1M Tris-HCl, 0.01M CaCl2, pH 8), at 37° C. for enzyme renaturation and subsequent proteolytic activity. The gels were washed three times, 5 min each, in deonized water prior to incubation for 1 h in Coomassie Brilliant Blue R-250. Images were obtained by ImageQuant LAS 500, GE Healthcare Life Sciences. Protease activity was visible as clear bands on a blue background. Images were obtained by ImageQuant LAS 500, GE Healthcare Life Sciences. Protease inhibition by phenyl methyl sulfonyl fluoride (PMSF) and EDTA was determined as described by Bertolini and Rohovec (Bertolini and Rohovec 1992).

### Results

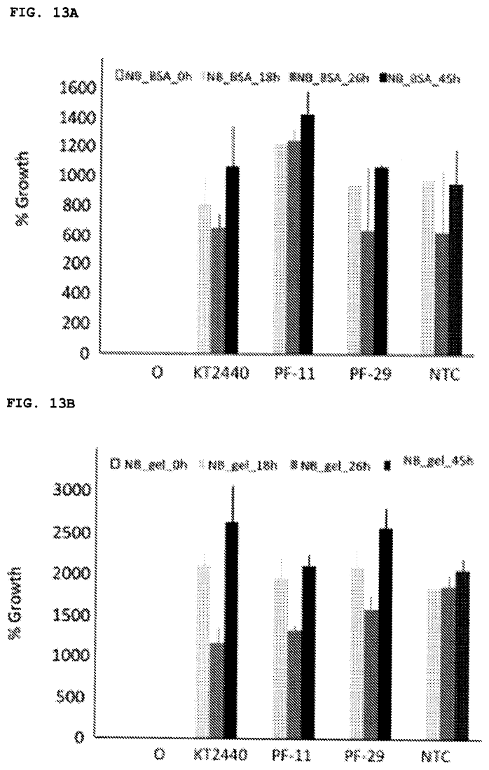

Our goal was to evaluate and compare the proteolytic activity of secreted enzymes from PF-11 and several others isolated strains. For that purpose, we have selected three basic media: M9 minimal medium for Pseudomonaceae (Miller 1972), a minimal medium designed for Pseudomonas described by (Prijambada, Negoro et al. 1995) with only slight differences to the first, and a rich medium defined to identify Pseudomonas aeruginosa, Nutrient Broth; we additionally adapted the first one either removing the nitrogen sources (ammonium and thiamine) designating it by M9-N or removing the sugar/energy supply (glucose), naming it M9-G (see Material and Methods). The NB, as a rich medium, should and has allowed indiscriminate growth of all strains independently on whether they produced extracellular proteases or not (FIG. 13A-B), as did any other tested media added of casaminoacids or skim-milk (data not shown) since those substrates did not require degradation to be internalized and used for growth. All the other media were designed aiming at growth limiting conditions, so that it would be mandatory for the supplemented substrates to be degraded in order for cultures to grow and record optical density readings. For historical reasons, as way to measure proteolysis we used as supplements skim-milk, caseine and casaminoacids (as a positive control for growth of all strains), but also other proteins already described for several biochemical approaches and protease production evaluation such as BSA, hemoglobin, and gelatin, all proteins which should not accomplish internalization into cells before being cleaved (see Material and Methods). HO was used as a negative control in the place of substrate.

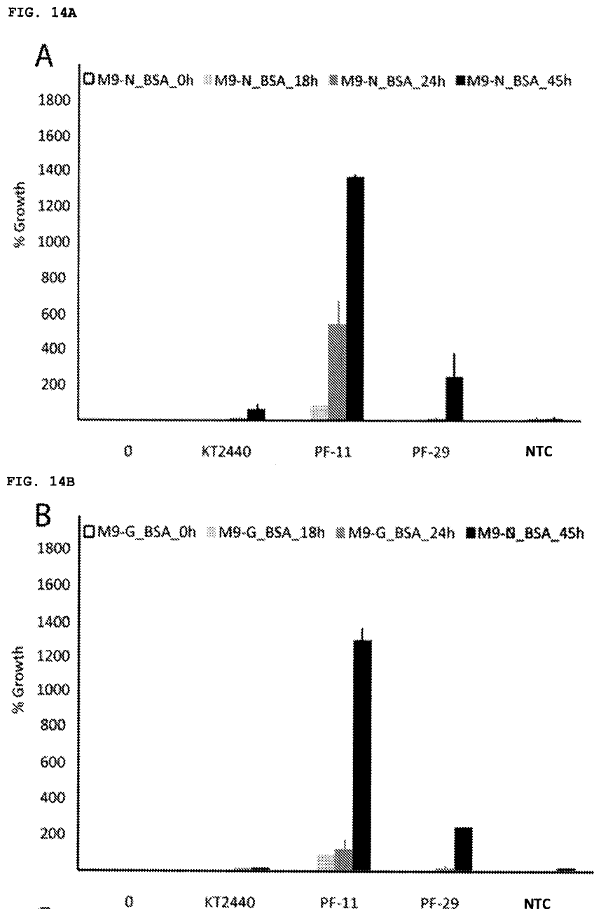

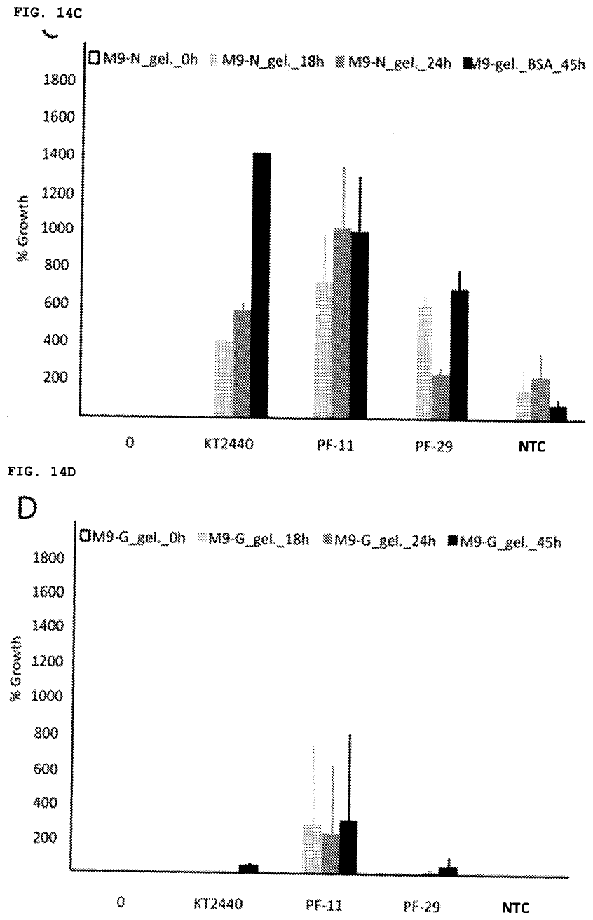

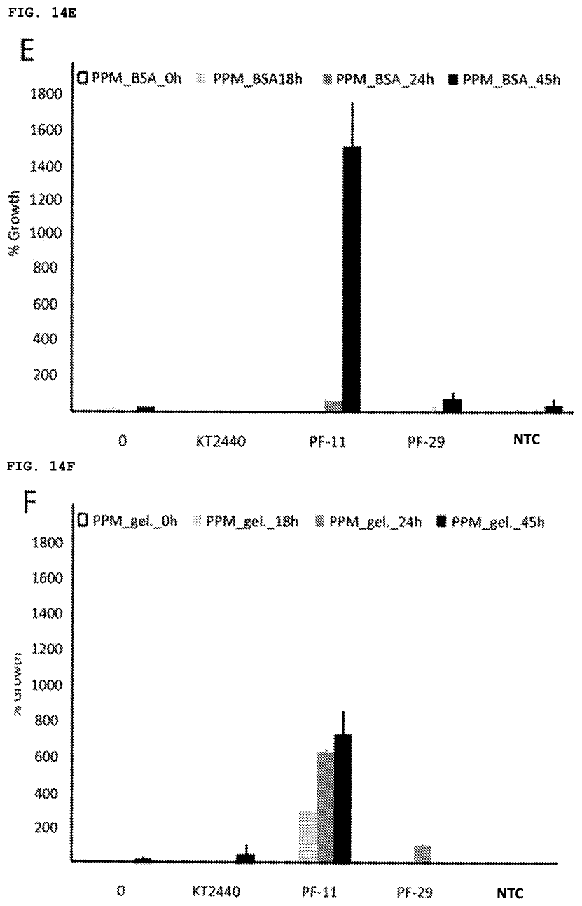

Media and supplements were permutated and evaluated against no strain, PF-29 as negative control, and PF-11 as positive control and finally against the P. putida KT2440 and P. aeruginosa reference strains, which were evaluated in the condition of unknown samples, although expecting the latter to have a positive response since P. aeruginosa are described as potent secretors of a number of active compounds. Total medium clearance could not be achieved by hemoglobin and casein dissolution. While retaining the light in its spectroscopic path and presenting an optical absorbance on their own, they masked the strains growth therefore preventing their use as proteinaceous supplements (data not shown). As for the rest of the options tested, the initial conditions were accomplished and results analysed. Several combinations of medium-proteinaceous supplement-M9-G+BSA, M9-G+gelatin, M9-N+gelatin, PPM+BSA, and PPM+gelatin-fulfilled the criteria proposed for the selection of protease producing strains (FIG. 14 A-F). Interestingly, while some of the supplements originated unspecific growth with a given medium they had perfect discriminatory power with others, as seen for gelatin in M9-N and M9-G, respectively (FIG. 14 C and D).

We selected 2 reference strains, Pseudomonas aeruginosa NTC27853 and Pseudomonas putida KT2440, and the environmental isolate PF-29 as negative control to better characterize the activity of the secretome of PF-11.

The proteolytic content of the bulk supernatants was analyzed by fluoresceine decay for all strains. Only PF-11 secreted proteins were able to degrade the fluorescent casein substrate for proteolytic content determination. The protease concentration normalized by the total secreted proteins is 100 μg/mg for PF-11. PF-11 strain presented clearly higher proteolytic content than the other strains. As negative control for the extracellular proteases production we used the PF-29 strain, previously tested and known not to present extracellular proteolytic activity.

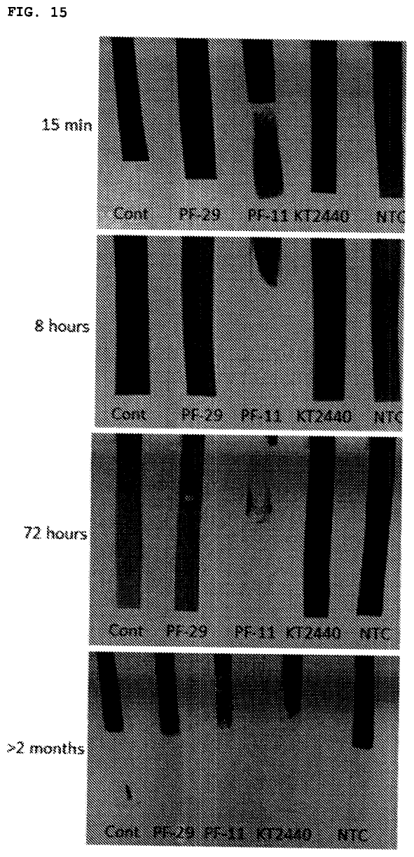

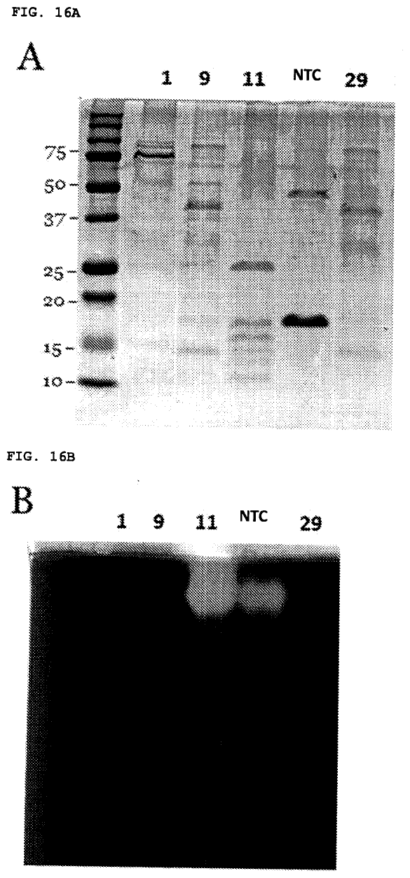

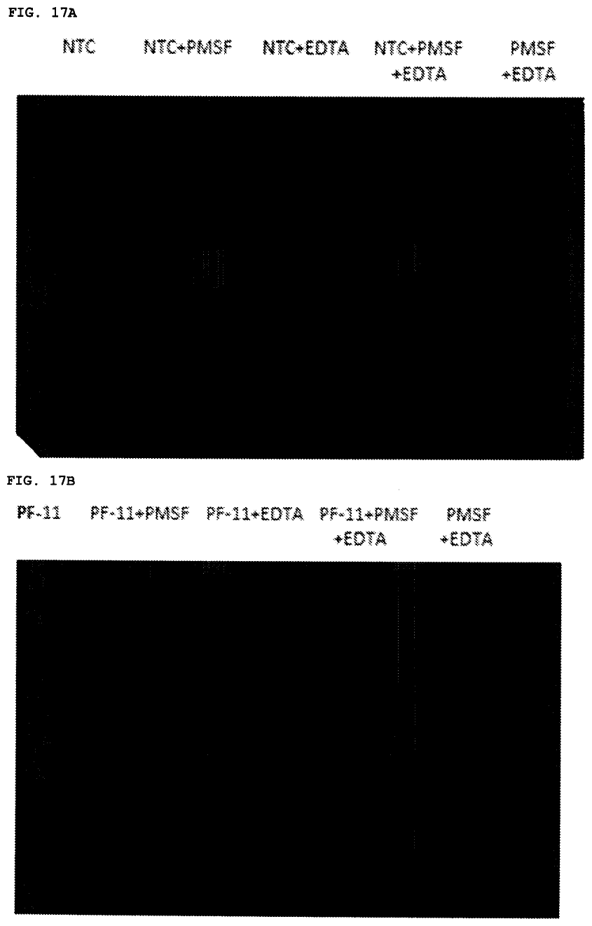

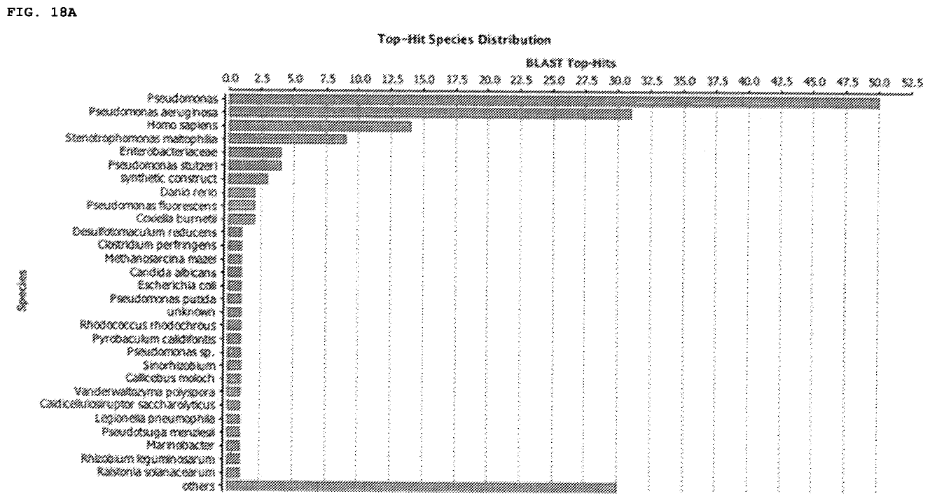

We compared proteolytic activity of PF-11, with reference strain Pseudomonas putida KT2440, isolate PF-29, identified previously as negative control, and isolates PF-9 and PF-22 that were previously identified as potential protease producers. For that, we performed an initial analysis by visual scrutiny of degradation of superficial gelatin layer of photofilms, after incubation to bacterial growth cultures. PF-11 bacterial broth reveals an outstanding proteolytic activity, revealed through the fast cleaning of the surface (FIG. 15).

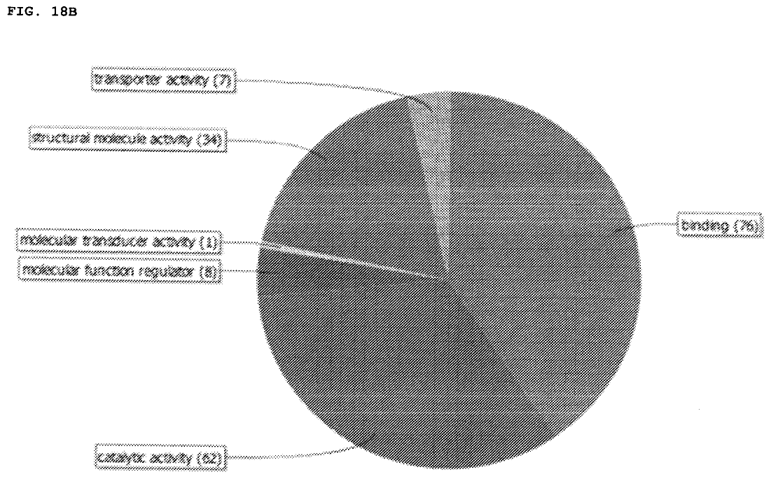

In terms of molecular approach, we analyzed the strains proteolytic activity by zymography, a sensitive and reliable method for the study of the proteases of different organisms, including Aer. salmonicida (Arnesen et al. 1995; Gudmundsdo′ ttir et al. 2003), and have further evaluated one more method to use as screening procedure. All tested strains were confirmed to secrete proteins as can be seen by their SDS PAGE protein profiles (FIG. 16A). Zymograms obtained on gelatin co-polymerized SDSPAGE allowed identifying different banding patterns among the tested strains (FIG. 16B). Of the strains evaluated, beside PF-11, only strain Pseudomonas aeruginosa NTC27853 exhibited one proteolytic activity band. As observed in the in vitro determination of the proteolytic content, the strain PF-11 presented the more intense bands, corresponding to a higher proteolytic activity.

The SDS PAGE protein profile presented proteins bands from 10 to 100 kDa and the more intense bands are not in the high molecular mass region. However the zymogram only presented proteolytic activities in the high molecular weight region. This fact could be due to some protein-protein complexes not being dissociated in low denaturing conditions required for zymography studies (Snoek-van Beurden and Von den Hoff 2005). Further evaluations of the type of proteases present in the extracellular protease producers were performed by selective inhibition of serine proteases by PMSF and of metallo-proteases by EDTA. When PF-11 and NTC were treated with 10 mM EDTA, their proteolytic activity was strongly inhibited, relatively to the non-treated control level, whereas that 10 mM PMSF presented significantly lower inhibitory effects (FIG. 17A and B). Therefore only about 10% of the protease activity in those secretomes is due to serine proteases, while about 50% is due to metallo-proteases.

### Example 6—Fungicide Effect of PF-11 Secretome

### Materials and Methods

MICs (Minimum Inhibitory Concentration) were determined according to CLSI standards (Clinical and Laboratory Standards Institute) M27-A3 (Reference Method for Broth Dilution Antifungal Susceptibility Testing of Yeasts; CLSI) and M38-A (Reference Method for Broth Dilution Antifungal Susceptibility Testing g of Filamentous fungi; CLSI). Filamentous fungi used were Aspergillus niger, Botrytis cinerea, Colletotrichum acutatum, Colletotrichum gloeosporioides and Fusarium oxysporum. Aspergillus niger is a fungus and one of the most common species of the genus Aspergillus. It causes a disease called black mould on certain fruits and vegetables such as grapes, apricots, onions, and peanuts, and is a common contaminant of food. Botrytis cinerea is an ecrotrophic fungus that affects many plant species, although its most notable hosts may be wine grapes. In viticulture, it is commonly known as botrytis bunch rot; in horticulture, it is usually called grey mould or gray mold. The fungus gives rise to two different kinds of infections on grapes. Colletotrichum acutatum is a plant pathogen. It is the organism that causes the most destructive fungal disease, anthracnose, of lupin species worldwide. Pathogenic strains of Fusarium oxysporum have an extremely broad range of hosts, and includes animals, ranging from arthropods to humans, as well as plants, including a range of both gymnosperms and angiosperms.

Yeasts used were Candida albicans and Candida glabrata. Candida albicans is a diploid fungus that grows both as yeastand filamentous cells and a causal agent of opportunistic oral and genital infections in humans, and candidal onychomycosis, an infection of the nail plate. Systemic fungal infections (fungemias) including those by C. albicans have emerged as important causes of morbidity and mortality inimmunocompromised patients (e.g., AIDS, cancer chemotherapy, organ or bone marrow transplantation)

### Results

PF-11 secretome displayed antifungal activity against Yeasts and Filamentous fungi Table 2. These results indicate that PF-11 secretome contains in its composition one or more active agents effective against fungi that can be used both for human health and for food plants, among all other relevant applications as fungicides.

TABLE 2

| | |
|---|---|
| Species | MIC (g/L) |

Filamentous fungi

| | |
|---|---|
| Aspergillus niger | 7.5 |
| Botrytis cinerea | 3.75 |
| Colletotrichum acutatum | 3.75 |
| Colletotrichum gloeosporioides | 3.75 |
| Fusarium oxysporum | 7.5 |

Yeasts

| | |
|---|---|
| Candida albicans | 3.75 |
| Candida glabrata | 3.75 |

Table 2—PF-11 secretome displayed antifungal activity against Yeasts and Filamentous fungi.

### Example 7—Larvicide/Insecticide Effect of PF-11 Secretome

### Materials and Methods

### Larvae

Late 3rd and/or early 4th instar larvae of the mosquitoes Culex theileri, Anopheles atroparvus and Anopheles gambiae were used. Mosquito colonies were boost in order to have an active number of mosquitoes producing larvae in sufficient numbers for the assays.

### Bioassay

Doses were planned by successive dilutions in order to integrate both 0 and 100% mortality. The assays were performed with 25 larvae in 250 ml of demineralized water per concentration, without food supply, in insectary controlled conditions, at 25-27° C. and 12 h light: 12 h dark photoperiod. Larval mortalities were recorded 24 h after. Demineralized water only was used as negative control. Assays were performed following WHO international guidelines to assess mosquito larvicides.

### Results

PF-11 Secretome can be used for insecticidal applications, since it exhibits the capability to control the proliferation of insects, by killing or preventing their development, through its use as chemical/biological compounds. We have observed insecticidal activity at low concentrations of the PF-11 secretome by killing mosquito larvae during their aquatic development phase, prior to metamorphosis into flying adults. PF-11 secretome presents 100% larval mortality at a concentration of 3.9 g/L after 24 hours.

### Example 8—Anti Marine Copepods Effect of PF-11 Secretome

### Materials and Methods

The assay consisted on incubating sea lice (Lepeophtheirus salmonis salmonis) larvae and copepodids with PF-11 secretome in order to evaluate the viability of the larvae and copepodids compared to a blank control. Sodium hypocholorite (NaoCl), known to kill the larvae within 40 min at 70 ppm, was used as a positive control.

### Larvae

Larvae were obtained by sedating salmon sea lice with Benzocain, harvesting egg strings with tweezers, and transferring the strings to a water bath placed in an incubator. Inside the incubator, the water was continuously changed throughout the incubation period. When the larvae were developed, they were removed from the incubator and used in the bioassay.

### Copepodids

Copepodids were developed from egg strings from sea lice as described for larvae.

### Bioassays

Seawater containing louse larvae and copepodids was placed in a petri dish and PF-11 secretome was added to the final pretended concentration. Larvae and copepodids in seawater with no added compounds were also used for comparison. The positive control was NaOCl at 70 ppm. The larvae and copepodids viability was checked over time. An evaluation of larvae viability was performed and compared to the viability in the blank controls. The viability in the pure seawater control and the medium blank control was monitored and compared and remained the same throughout the experiment.

### Results

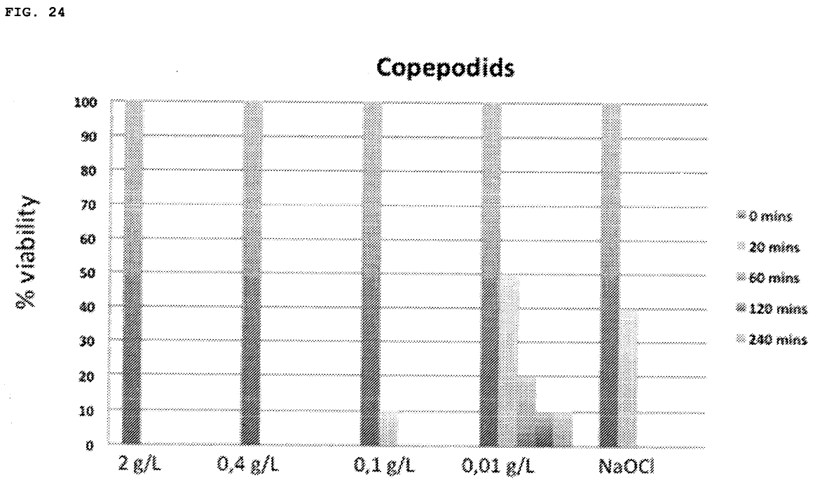

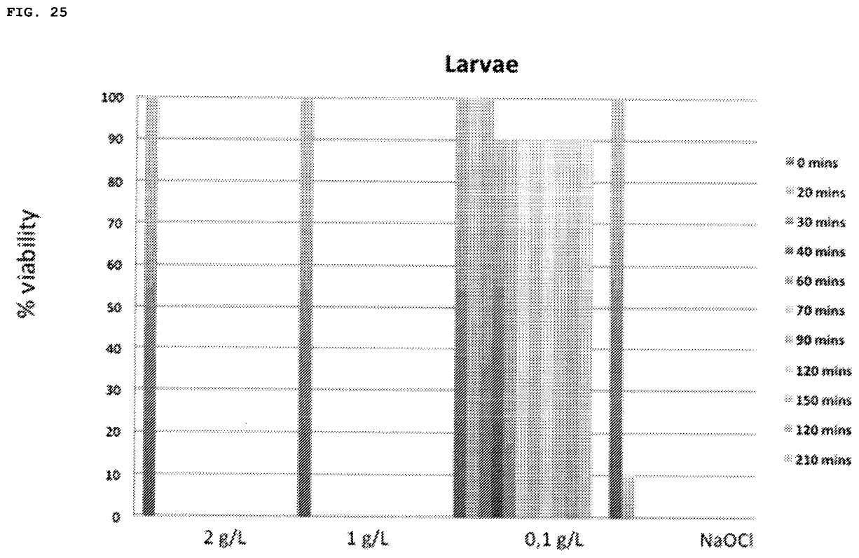

At the concentration range used PF-11 secretome displayed substantially greater activity against sea lice larvae (FIG. 24) and Copepodids (FIG. 25) than the positive control NaOCI at 70 ppm, killing 100% of sea lice larvae and Copepodids before 20 mins, the required time for NaoCl become 100% effective. These results indicate that PF11 secretome is effective against sea lice and therefore displays anti-parasitic activity.

### DISCUSSION

### Antimicrobial Resistance

The emergence of antimicrobial resistance to practically all antibiotics in use nowadays is rapidly leading to a situation where no efficient therapy for bacterial infections will soon be available. Any newly introduced antibiotic, used medically, has been overcome in less than one year by the detection of bacterial resistance that inhibits its effect. The search for new antimicrobial compounds must therefore integrate the need to find efficient antibiotics that will not foster new resistance mechanisms in bacteria or select for resistance processes that already exist.

Environmental bio-prospection of active biological compounds is an attractive strategy for the development of such new natural tools, in this case for the control of bacterial proliferation. Environmental microorganisms are continuously under the pressure of changing surroundings, which induces an active selection of highly resistant bacterial cells that must possess a rich array of molecular responses to cope with external alterations and compete against neighboring microorganisms for nutrients. Bacteria have been developing tools to overpower their competitors for millions of years, ensuring their own survival and access to nutrients by inhibiting the growth of neighbouring microorganisms or even destroying them. Bacteria are natural producers of extracellular molecules that have been successfully used in applications in the most diverse areas (antibiotics production, biochemical processes, food industry, etc.) (Wilhelm et al., Wu and Chen et al., Liu and Li 2011, Pontes et al.).

Antimicrobial peptides (AMP) are excellent candidates for infection control, as they rapidly disrupt bacterial membranes, which confers them broad-spectrum activity against Gram+ and Gram-species. Notably, although AMPs are widely distributed in nature and bacteria have been exposed to these molecules for millions of years, widespread resistance has not been reported (Fjell et al. 2011). Given the increase of microbial resistance to classic antibiotics, the use of AMPs stands out as a valuable alternative for future therapy of bacterial infections. AMPs are produced by all species of life and represent key components of the innate immune system, providing a fast acting weapon against invading pathogens including bacteria, fungi, and yeast (Boman, 1995; Hancock et al., 2006; Selsted and Ouellette, 2005; Zasloff, 2002). AMPs can rapidly penetrate, permeate and destroy membranes (Ludtke et al., 1996; Pouny et al., 1992; Shai, 2002) causing irreversible cell damage in contrast to conventional antibiotics with which they have no cross-resistance (Vooturi et al.); the irreversibility of their action reduces the probability of microbial resistance emergence (Zasloff, 2002). Eukaryotic AMPs are in general large-spectrum antimicrobials, but most are toxic to both bacteria and eukaryotic cells, invalidating their direct use (Asthana 2004). The high cytotoxycity and low bioavailability of eukaryotic AMPs have hampered clinical applications so far, generally due to proteolytic degradation of the peptides or their aggregation that occurs in high concentrations necessary for efficiency (Giuliani 2008). In contrast, AMPs produced by bacteria, such as lipopeptides or peptidolipids, are selective and show lower toxicity to animals (Parisien 2008). Lipopeptides are only produced in bacteria and fungi, and possess as potent antimicrobial activity as well as surfactant properties. Nevertheless, native lipopeptides are non-cell-selective and can therefore be toxic to mammalian cells, too. Despite this, daptomycin, a member of this family, is active only toward Gram-positive bacteria and was recently approved by the Food and Drug Administration (FDA) for the treatment of complicated skin infections (Department of Health and Human Services, 2003). Peptidolipids are also under investigation due to their capability to destroy or remove phytopathogens. Making use of their surfactant properties, they can either stimulate the attachment and/or detachment of bacteria from surfaces, bacterial biofilm development and maintenance (O'Toole et al., 2000), and bacterial motility, cellular communication and nutrient access (Al-Tahhan et al., 2000; Garcia-Junco et al., 2001). Even though AMPs from bacterial origin have been studied, with limited success regarding application, most attempts to transfer these approaches have been focused on eukaryotic AMPs. However, the impact that environmental bacteria have on the processes of AMR and their largely undiscovered variety opens a window of opportunity for the research of new antimicrobials.

In Example 3, a heterogeneous collection of environmental Pseudomonas putida strains, collected in the course of previous studies through resistance to antibiotics (Meireles 2013), with strong adaptive skills, was used to screen the potential of secreted natural compounds for microbiological growth control. Emphasis was placed on secreted molecules, since it should lead to compounds more stable in a variety of temperatures and conditions, and especially to molecules naturally used to affect the growth of neighboring competitors, with a proof of efficiency provided by the survival of the producing bacteria. Pseudomonas spp. is generally widespread in the environment and persistent in highly polluted areas. They have furthermore been shown to actively secrete molecules related to bacterial communication like homoserine lactone auto-inducers in quorum-sensing communication (Roy et al.), different types of siderophores like pyoverdine or pyocyanin (Nestler et al.), exopolysaccharides and several different enzymes, including extracellular proteases (Wilhelm et al.).

A collection of environmental P. putida isolates was used to screen for antimicrobial compounds secretion. The secretomes of a set of seven isolates and a P. putida reference strain were used to determine their antimicrobial activity. Some secretomes showed potential for bacterial growth inhibition, however the compounds secreted by strain PF-11 revealed a surprising and outstanding impact on the growth prevention of other bacteria (FIG. 1A-C). However, it presented no inhibition of the growth of P. aeruginosa, a closely related genre to P. putida, also widely ubiquitous and known for its adaptation skills. In contrast, strong growth inhibition of the growth of E. coli and S. aureus was determined, showing impact both on Gram-negative and -positive strains. These reference strains used as representatives of a genre, showed the strong potential of the secretome of PF-11 to contain compounds susceptible to control pathogenic E. coli and S. aureus strains. The growth of other P. putida strains, including isolates and KT2440, was also strongly impaired (FIG. 2).

Remarkably, when tested on its own growth, the secretome of PF-11 acted as a growth stimulant, revealing the presence of specific compounds that stimulate that strains replication, in a specific intra-strain communication. Neither of the other P. putida strains or even P. aeruginosa showed such behaviour, emphasizing the uniqueness of PF-11 strain. This bacterium clearly presents the features of a successful environmental survivor, with the tools to strongly inhibit competitors and the asset to overstimulate the replication of its siblings.

The separation of the secretome of PF-11 strains by molecule size exclusion generated a fraction containing peptides and small compounds (size lower than 10 kDa) and another fraction with larger compounds (proteins, including enzymes, and others). Data in FIG. 3 confirms the absence of impairment of P. aeruginosa growth for either fraction, while E. coli and P. putida are essentially inhibited by the protein fraction. S. aureus is strongly inhibited by any of the fraction, however the peptidic fraction has clearly a stronger anti-staphylococcal effect. These results show, that even though the secretome of PF-11 antimicrobial properties, it is supported by different compounds or molecules, and affects distinctly different bacteria. Therefore, this secretome contains a mixture of different elements, in distinct combinations or maybe even by themselves, which inhibit bacterial proliferation and can target distinct bacterial genres. Hence, the compounds secreted by strain PF-11 revealed an enlarged potential for antimicrobial applications, suggesting the existance of several molecules of interest for diverse applications.

A general characterization of the PF-11 secretome in terms of composition revealed the presence of peptides in outstanding concentration (FIG. 4), surfactant molecules, eventually lipopeptides, (FIG. 5) and degradative enzymes (FIG. 6). All these sorts of compounds are secreted by the bacteria in large amounts, in a clear outstanding behaviour towards P. putida KT2440, and confirming the richness of this secretome in terms of potential antimicrobial compounds.

To clear the path for future applications, the secretome of PF-11 strain was collected and concentrated by liophilization, in order to maintain the biological activity of the compounds. The results obtained with E. coli and S. aureus previously were validated and the impact on pathogenic strains E. coli 0157 and methicilin-resistant S. aureus (MRSA) ATCC 33591 was established. Antimicrobial compounds are found in the different tested fractions of this secretome, however the peptidic fraction contains the molecules with a higher antimicrobial impact on distinct bacteria, probably antimicrobial peptides, eventually carrying surfactant activity.

### Antifouling

In marine environments, after such initial stage of microfouling, a phase of macrofouling eventually occurs consisting on the fixation of seaweeds, barnacles and other marine invertebrates, causing functional and maintenance problems to immersed artificial structures such as ship hulls, aquaculture net cages, seawater intake pipes or offshore platforms (Railkin et al.). Microfouling on ship hulls increases frictional drag and is responsible, by itself, for an increase of 21% in fuel consumption in shipping, while the impact of macrofouling can cause energy losses close to 86% (Schultz et al.).

Around 90% of the world trade today is based on international shipping by sea (International Chamber of Shipping). The financial impact of marine biofouling on international maritime transportation costs is considerable, and has led to the exponential need of research in antifouling technologies, in the frame of a global industry estimated in US$4 billion per year (Dafforn et al.). Such a heavy impact of biofouling on the competitiveness of this industry has led to the development and implementation of antifouling solutions, since the origins of maritime transportation. The coatings of vessel hulls aim at reducing corrosion and preventing biological adherence.

Toxic compounds, such as copper and tributyltin, have been added to the paints used in this process, and prevented with success the formation of biofouling by their continuous release to the surrounding sea (Yebra et al.). However, the widespread use of such substances, especially tributyltin, led to its accumulation in the environment, generating worldwide concern due to its non-specificity and resulting toxic impact on marine communities (Thomas et al.). As a result, the International Maritime Organisation banned tributyltin-based paints from use in 2003, which resulted in the lack of efficient antifouling solutions (International Maritime Organisation, London). Copper-based paints still remain in use and new non-toxic silicone-based coatings have been developed and implemented, however their use is strongly regulated and furthermore their efficiency is reduced (Townsin et al.). It is therefore understandable that research of novel and natural antifouling compounds has been developing and remains the most promising approach to implement non-toxic and efficient methodologies to control biological proliferation, however no solutions have been commercially implemented with success yet (Dafforn et al.).

Thus, research has been driven to the identification of enzymes, without toxic impact on the environment, that are able to prevent or disrupt the formation of these adhesion structures (Leroy et al., Pettitt et al.). Among degradative or anti-proliferative compounds with potential impact on biofouling, the degradative enzymatic activity of proteases has been suggested as a promising path, since the core of the adhesive glues or molecules used by biological species, from bacteria to lower eukaryotes, is essentially proteic (Rawlings et al.). Mixtures of proteases have been shown to inhibit the settlement of Ulva zoospores, Balanus amphitrite cyprid larvae and Bugula neritina (Pettitt et al., Dobretsov et al.), and such activity was confirmed to be due to the reduction of adhesive effectiveness, probably through the degradation of peptide-based adhesive compounds (Aldred et al.). Furthermore, an antifouling effect related to proteases was established when such enzymes were incorporated into water-based paints (Dobretsov et al.). In addition, proteins constitute an important part of biofilm matrices and proteases can be efficient in disrupting these structures, as observed for Pseudoalteromonas biofilm formation (Leroy et al.).

Environmental bio-prospection of active biological compounds is an attractive strategy for the development of novel biotechnological tools. Bacteria are natural producers of extracellular molecules that have been successfully used in applications in the most diverse areas (antibiotics production, biochemical processes, food industry, etc) (Wilhelm et al., Pontes et al.). Environmental Enterobacteriaceae, especially Pseudomonaceae, are known to secrete extracellular proteases into the surrounding medium. These secreted proteases are sometimes factors of virulence contributing for infective strategies, like the alkaline protease and elastase produced by Pseudomonas aeruginosa (Liu 1974) but have also been used as anti-inflammatory agents, like a metalloprotease produced by Serratia sp. strain E-15 (Nakahama et al.). Aeromonas hydrophila, an opportunistic human and fish pathogen, was found to produce two distinct types of extracellular proteases, a temperature-stable metalloprotease and a temperature-labile serine protease (Leung et al.).

Environmental strains, especially when located at the soil surface or near river shores, are continuously under the influence of unstable surroundings. The regular daily variations of temperature, humidity, presence or absence of light or nutrients, among others, tend to enforce a selective pressure on these bacteria that is increased when anthropogenic factors are considered. The latter can vary from slow continuous contaminations, of different sorts (chemicals, pesticides, faecally contaminated sewage waters, etc.) to sudden industrial toxic discharges. All these factors induce an active selection of highly resistant specialized bacterial cells, and simultaneously, bacteria that must possess a large array of responses to cope with such large variety of external pressures. Such harsh conditions are reflected on environmental strains highly successful on acquiring nutrients from different origins and through different degradation mechanisms, on resisting to external conditions variation but also highly competitive against neighboring microorganisms, making use of authentic arsenals in a silent war for survival. The mechanisms involved in these processes and the biomolecules produced are the result of billions of years of evolution directly tested in real situations, with their efficiency proven by the survival of the carrying species. Such communities become therefore reservoirs of excellence to screen for molecules to be used in applications leading to biocontrol of organisms.

Pseudomonas putida, a Pseudomonaceae mostly found in soil, is a saprotrophic microorganism, capable of chemoheterotrophic extracellular digestion of decayed matter. As one of the most well adapted environmental bacteria, P. putida possesses an arsenal of adaptative skills genetically enclosed, that allow not only the active capture of surrounding nutrients, but also the control of other competitors, bacteria and fungi, by affecting their growth through the secretion of toxic or anti-proliferative compounds (Gjermansen et al., Tsuru et al.).

In Example 4, a selection was performed to detect environmental Pseudomonaceae strains able to produce extracellular compounds relevant for biotechnological applications as antifouling agents, naturally produced and non-toxic to environmental communities. Pseudomonas spp. strains are widespread in the environment and persistent in highly polluted areas (Madigan et al.). They have furthermore been shown to actively secrete molecules related to bacterial communication like homoserine lactone auto-inducers related to quorum-sensing (Charlton et al., Huang et al.), different types of or pyocyanin siderophores like pyoverdine (Meyer et al.), exopolysaccharides and several different enzymes, including extracellular proteases (Liu 1974). The possibility to collect secreted biomolecules presents several interesting advantages when biotechnological applications are considered. Not only their recovery in large quantities, necessary for analytical characterization and use, is facilitated, but also being molecules usually secreted to the environment, they should in principle be more stable and therefore more resistant to spontaneous degradation than intracellular molecules.

Among the tested strains, Pseudomonas PF-11 was established as an exceptional protease-secreting strain, producing a protease, or more probably a mixture of proteases, able to degrade casein, total E. coli protein extracts and the adhesives secreted by sea urchins. Furthermore, the activity detected remained fairly stable in a large interval of temperatures and presented a very low turnover rate. This proteolytic activity depends on the presence of secreted proteases in the supernatant of PF-11 cell cultures. As described above, proteases have been consistently considered as good enzymatic candidates for antifouling coating applications.

In addition, both the supernatant mixture produced by this strain and the bulk culture by itself, were able to disrupt marine biofilms and sea urchin adhesive footprints attached to glass substrates. To a large extent, these effects can be attributed to the presence of proteases in the solutions used, since the dismantlement of natively structured proteins is sufficient to abolish any disruption. Indeed, the protein based adhesive structures, either of the sea urchins or of the adherent mechanisms of the diverse microorganisms present in the marine biofilms, constitute an ideal target for proteolysis, and subsequent disruption of the fouling. However, other regulatory elements might integrate this secreted mixture and in combination contribute to such a strong impact on the adhesive integrity. The secretome of Pseudomonas PF-11 apparently constitutes a rich and complex mixture of extremely relevant potential antifouling compounds.

The strain Pseudomonas PF-11, isolated from the environment, is able to secrete a concentrated mixture of proteases and probably other compounds, which promote strong antifouling effects, both on micro- and macrofouling events. The characterization of the antifouling components of this secreted mixture is required and is the logical in-progress continuation of this research, in order to determine the active players involved in the biofouling removal. Its perceived potential goes far beyond the proteolysis activity detected. The identification of the molecules involved in this process will not only shed more light on the mechanisms of biological adhesion, but will certainly contribute with a novel set of bioactive molecules, environmental-friendly, that may become part of the solution for several biofouling hazards, especially in marine biofouling elimination strategies.

### Proteases

Proteases are enzymes that perform proteolysis of other proteins or oligopeptides, hydrolyzing the peptidic bonds between sequential amino acids. The nucleophylic attack may occur either associated with specific amino acids, within the peptide chains by endopeptidases, or it can be unspecific, at the extremity of the proteins, by exopeptidases. Accordingly, the substrates are partially degraded either into shorter chains or oligopeptides, or completely, releasing their amino acidic building blocks (Barrett 2001). These biocatalyzers are defined as acidic, neutral or alkaline, consistent with the pH range where they exert their activity (Gupta, Beg et al. 2002). According to their mechanisms of catalysis, substrates specificity, and even protein small molecule inhibitors, proteases can also be classified as asparagine peptide lyases, or aspartic, cysteine, glutamic, serine, threonine, and metallo or unknown catalytic type peptidases (Rawlings, Barrett et al. 2012). So far, the best studied of all have been extracellular bacterial proteases and within those, the serine and metallo proteases (Wu et al.).

The world demand for biological catalysts is expected to reach $7 billion in 2013. Proteases represent today one of the major groups of industrial enzymes and a number of detergent stable proteases have been isolated and characterized because of their widespread use (GCI 2009). Extracellular bacterial proteases (EBP) present several characteristics that are unique in the context of bioengineering and industry: they are translated as pre-propeptides, and secreted by the cell (therefore becoming naturally accessible in the growth medium, averting extraordinary methods to acquire them and further allowing cultures to be maintained while recovering the compounds of interest), and furthermore they only become active outside the cell (therefore not becoming toxic upon overexpression or reducing the culture growth and ability to be maintained due to excessive accumulation). The enzymes' active form is then achieved either by auto-processing through intramolecular chaperones, which upon maturation are cleaved (Kessler and Ohman 2004; Gao, Wang et al. 2010) or aided by other proteases which act as their regulators (Kessler, Safrin et al. 1998). Besides these aspects, extracellular proteases usually present optimum temperature, pH (and pI), and ionic strength dependent on the environment where the isolates grow, so it is possible to narrow screenings for intended activities towards certain isolate strains according to their origin as it is possible to induce cultures and proteins adaptation to grow and function in extreme conditions creating man-induced evolutionary pressures towards certain environments, solvents, or temperatures. Moreover secreted biocatalysts usually naturally present interesting thermal, pH, and even salt stabilities, since they are not buffered outside the cells (Wu and Chen et al. 2011).

But if proteases are ubiquitous, most of them are not industry material either due to the level of expression or to downstream processing requirements and consequent cost of production, as due to the lack of substrate specificity, or solvent stability/activity; this is to say to their (in) adaptability to the conditions for their intended use (Gupta, Beg et al. 2002). Nevertheless, microbial proteases represent most of the industrial proteases production since before 1999, and their market only tends to grow (Godfrey and West 1996; Kumar and Takagi 1999).

### REFERENCES

Abarzua, S. and S. Jakubowski, Biotechnological investigation for the prevention of biofouling I. Biological and biochemical principles for the prevention of biofouling. Marine Ecology Progress Series 1995. 123: p. 301-12.
Aldred, N. and A. S. Clare, The adhesive strategies of cyprids and development of barnacle-resistant marine coatings. Biofouling, 2008. 24 (5): p. 351-63.
Al-Tahhan, R. A., et al. 2000. Rhamnolipid-induced removal of lipopolysaccharide from Pseudomonas aeruginosa: effect on cell surface properties and interaction with hydrophobic substrates. Appl Environ Microbiol 66:3262-8.
Angilletta, M. J., Jr., R. B. Huey, and M. R. Frazier, Thermodynamic effects on organismal performance: is hotter better? Physiol Biochem Zool, 2010. 83 (2): p. 197-206.
Arnesen, J. A. et al. (1995), Partial purification and characterization of extracellular metalloproteases from Aeromonas salmonicida ssp. salmonicida. J Fish Dis 18, 283-295.
Asthana, N. et al. (2004), Dissection of antibacterial and toxic activity of melittin: a leucine zipper motif plays a crucial role in determining its hemolytic activity but not antibacterial activity. J Biol Chem 279, 55042-55050.
Barrett, A. (2001). Proteolytic enzymes: nomenclature and classification. In: Beynon R, Bond JS (eds) Proteolytic enzymes. A practical approach, 2nd edn. Oxford University Press, Oxford.” 1-21.
Bertolini, J. M., Rohovec, J. S. (1992). “Electrophoretic detection of proteases from different Flexibacter columnaris strains and assessment of their variability”. Diseases of Aquatic Organisms 12:121-128
Boman, H. G., Peptide antibiotics and their role in innate immunity. Annu Rev Immunol. 1995; 13:61-92.
Boucher, H. W., G. H. Talbot, J. S. Bradley, et al. Bad bugs, no drugs: no ESKAPE! An update from the Infectious Diseases Society of America. Clin Infect Dis. 2009; 48:1-12.
Charlton, T. S., et al., A novel and sensitive method for the quantification of N-3-oxoacyl homoserine lactones using gas chromatography-mass spectrometry: application model bacterial biofilm. Environ Microbiol, 2000. 2 (5): p. 530-41.
da Costa, G. et al. (2011), Beyond genetic factors in familial amyloidotic polyneuropathy: protein glycation and the loss of fibrinogen's chaperone activity. PLOS One 6 (10): e24850.
Dafforn, K. A., J. A. Lewis, and E. L. Johnston, Antifouling strategies: history and regulation, ecological impacts and mitigation. Mar Pollut Bull, 2011. 62 (3): p. 453-65.
Department of Health and Human Services. Center for Drug Evaluation and Research Approval Package For: Application Number 21-572-Approval Letter(s). 2003.
Dobretsov, S., et al., Novel antifoulants: inhibition of larval attachment by proteases. Mar Biotechnol (NY), 2007. 9 (3): p. 388-97.
ECDC/EMA joint technical report. The bacterial challenge: time to react. (September 2009).
Fjell, C. D. et al., Designing antimicrobial peptides: form follows function. Nat Rev Drug Discov. 2011 Dec. 16; 11 (1): 37-51.
FAO/OIE/WHO. Joint FAO/OIE/WHO Expert Workshop on Non-Human Antimicrobial Usage and Antimicrobial Resistance: Scientific assessment-Draft Report. Geneva, Dec. 1-5, 2003.
Gaby, W. L., C. Hadley, Practical laboratory test for the identification of Pseudomonas aeruginosa. J Bacteriol. 1957 September; 74 (3): 356-8.
Gao, X., J. Wang, et al. (2010). Structural basis for the autoprocessing of zinc metalloproteases in the thermolysin family. Proc Natl Acad Sci USA 107 (41): 17569-17574.
Garcia-Junco, M., E. De Olmedo, and J. J. Ortega-Calvo. 2001. Bioavailability of solid and non-aqueous phase liquid (NAPL)—dissolved phenanthrene to the biosurfactant-producing bacterium Pseudomonas aeruginosa 19SJ. Environ Microbiol 3:561-9.
GCI, G. C. I. (2009). World Enzymes Market-Industry Study with Forecasts for 2013 & 2018. Freedonia.
Giuliani, A. et al., Antimicrobial peptides: natural templates for synthetic membrane-active compounds. Cell Mol Life Sci. 2008 August; 65 (16): 2450-60.
Gjermansen, M., et al., Characterization of starvation-induced dispersion in Pseudomonas putida biofilms: genetic elements and molecular mechanisms. Mol Microbiol, 2010. 75 (4): p. 815-26.
Godfrey, T. and S. West (1996). Introduction to industrial enzymology. In: Godfrey, T., West, S. (eds.) Industrial enzymology, 2nd edn. MacMillan Press, London. 1-8.
Gudmundsdottir S., Subtyping of Salmonella enterica serovar typhimurium outbreak strains isolated from humans and animals in Iceland. J Clin Microbiol. 2003 October; 41 (10): 4833-5.
Gupta, R., Q. K. Beg, et al. (2002). Bacterial alkaline proteases: molecular approaches and industrial applications. Appl Microbiol Biotechnol 59 (1): 15-32.
Hancock, R. E. et al., Antimicrobial and host-defense peptides as new anti-infective therapeutic strategies. Nat Biotechnol. 2006 December; 24 (12): 1551-7.
Hajna, A. A., Triple-Sugar Iron Agar Medium for the Identification of the Intestinal Group of Bacteria. J Bacteriol. 1945 May; 49 (5): 516-7. Huang, Y. L., et al., Presence of acyl-homoserine lactone in subtidal biofilm and the implication in larval behavioral response in the polychaete Hydroides elegans. Microb Ecol, 2007. 54 (2): p. 384-92.
International Chamber of Shipping (ICS), ICS_ICS&ISF, 2009, available at marisec.org/shippingfacts/home/, Jan. 1, 2012).
International convention on the control of harmful antifouling systems on ships. International Maritime Organisation, London.
Kaplan, W. et al., Priority Medicines for Europe and the World, Geneva: World Health Organization (2004).
Kessler, E. and D. E. Ohman (2004). Pseudolysin. In: Barret, A. J., Rawlings, N. D. (eds.) Handbook of proteolytic enzymes, vol. 1, 2nd edn. Elsevier, Amsterdam. 401-409.
Kessler, E., M. Safrin, et al. (1998). Elastase and the LasA protease of Pseudomonas aeruginosa are secreted with their propeptides. J Biol Chem 273 (46): 30225-30231.
Kumar, C. G. and H. Takagi (1999). Microbial alkaline proteases: from a bioindustrial viewpoint. Biotechnol Adv 17 (7): 561-594.
Laemmli, U. K., Cleavage of structural proteins during the assembly of the head of bacteriophage T4. Nature. 1970 Aug. 15; 227 (5259): 680-5.
Lamy E., et al., Changes in mouse whole saliva soluble proteome induced by tannin-enriched diet. Proteome Sci. 2010 Dec. 15; 8:65.
Leroy, C., et al., Effects of commercial enzymes on the adhesion of a marine biofilm-forming bacterium. Biofouling, 2008. 24 (1): p. 11-22.
Leung, K. Y. and R. M. Stevenson, Characteristics and distribution of extracellular proteases from Aeromonas hydrophila. J. Gen. Microbiol., 1988. 134: p. 151-160
Liu, M. and A. Li, (Quorum sensing involved in the regulation of secondary metabolism in streptomycetes—a review). Wei Sheng Wu Xue Bao, 2011. 51 (5): p. 571-8.
Liu, P. V., Extracellular toxins of Pseudomonas aeruginosa. J Infect Dis, 1974. 130 Suppl (0): p. S94-9.
Livermore, D. M., British Society for Antimicrobial Chemotherapy Working Party on The Urgent Need: Regenerating Antibacterial Drug Discovery and Development. Discovery research: the scientific challenge of finding new antibiotics. J Antimicrob Chemother. 2011; 66:1941-1944.
Ludtke, S. J. et al., Membrane pores induced by magainin. Biochemistry. 1996 Oct. 29; 35 (43): 13723-8.
Madigan, M. and J. Martinko, Brock Biology of Microorganisms (11th ed.), ed. M. a. M. Madigan, J. 2005: Prentice Hall.
Martínez, J L. Antibiotics and antibiotic resistance genes in natural environments. Science. 2008 Jul. 18; 321 (5887): 365-7
McKerrow, J. H., V. Bhargava, et al. (2000). A functional proteomics screen of proteases in colorectal carcinoma. Mol Med 6 (5): 450-460.
Meireles C, Costa G, Guinote I, Albuquerque T, Botelho A, Cordeiro C, Freire P. (2013) Pseudomonas putida are environmental reservoirs of antimicrobial resistance to β-lactamic antibiotics. World J Microbiol Biotechnol. 2013 July; 29 (7): 1317-25.
Meyer, J. M., et al., Siderophore typing, a powerful tool for the identification of fluorescent and nonfluorescent pseudomonads. Appl Environ Microbiol, 2002. 68 (6): p. 2745-53.
Miller, J. H., Experiments in Molecular Genetics. Cold Spring Harbor Laboratory Press, Cold Spring Harbor, NY, 1972.
Nakahama, K., et al., Cloning and sequencing of Serratia protease gene. Nucleic Acids Res, 1986. 14 (14): p. 5843-55.
Nakazawa, T. and T. Yokota, Benzoate metabolism in Pseudomonas putida (arvilla) mt-2: demonstration of two benzoate pathways. J Bacteriol, 1973. 115 (1): p. 262-7.
Nelson, K. E., et al., Complete genome sequence and comparative analysis of the metabolically versatile Pseudomonas putida KT2440. Environ Microbiol, 2002. 4 (12): p. 799-808.
Nestler, H. P. and A. Doseff, A two-dimensional, diagonal sodium dodecyl sulfate-polyacrylamide gel electrophoresis technique to screen for protease substrates in protein mixtures. Anal Biochem, 1997. 251 (1): p. 122-5.
Ołdak E., E. A. Trafny. Secretion of proteases by Pseudomonas aeruginosa biofilms exposed to ciprofloxacin. Antimicrob Agents Chemother. 2005 August; 49 (8): 3281-8.
Olsen, S. M., et al., Enzyme-based antifouling coatings: a review. Biofouling, 2007. 23 (5-6): p. 369-83.
O'Toole, G. et al. 2000. Biofilm formation as microbial development. Annu Rev Microbiol 54:49-79.
Palleroni, N. J., The Pseudomonas story. Environ Microbiol, 2010. 12 (6): p. 1377-83.
Parisien, A. et al. (2008), Novel alternatives to antibiotics: bacteriophages, bacterial cell wall hydrolases, and antimicrobial peptides. Journal of Applied Microbiology, 104:1-13.
Payne, D. J., M. N. Gwynn, D. J. Holmes, D. L. Pompliano. Drugs for bad bugs: confronting the challenges of antibacterial discovery. Nat Rev Drug Discov. 2007; 6:29-40.
Pettitt, M. E., et al., Activity of commercial enzymes on settlement and adhesion of cypris larvae of the barnacle Balanus amphitrite, spores of the green alga Ulva linza, and the diatom Navicula perminuta. Biofouling, 2004. 20 (6): p. 299-311.
Pontes, D. S., et al., Lactococcus lactis as a live vector: heterologous protein production and DNA delivery systems. Protein Expr Purif, 2011. 79 (2): p. 165-75.
Pouny, Y., et al., Interaction of antimicrobial dermaseptin and its fluorescently labeled analogues with phospholipid membranes. Biochemistry. 1992 Dec. 15; 31 (49): 12416-23.
Priest, F. G., Synthesis and secretion of extracellular enzymes by bacilli. Microbiol Sci, 1985. 2 (9): p. 278-82.
Prijambada, I. D., et al. Emergence of nylon oligomer degradation enzymes in Pseudomonas aeruginosa PAO through experimental evolution. Appl Environ Microbiol. 1995 May; 61 (5): 2020-2
Railkin, A. I., Marine biofouling: colonization processes and defences. Boca Raton: CRC Press LLC, 2004.
Rawlings, N. D., F. R. Morton, and A. J. Barrett, MEROPS: the peptidase database. Nucleic Acids Res, 2006. 34 (Database issue): p. D270-2.
Rawlings, N. D., A. J. Barrett, et al. (2012). “MEROPS: the database of proteolytic enzymes, their substrates and inhibitors.” Nucleic Acids Res 40 (Database issue): D343-350.
Roy, K., et al., Adhesin degradation accelerates delivery of heat-labile toxin by enterotoxigenic Escherichia coli. J Biol Chem. 286 (34): p. 29771-9.
Sambrook, J., T. Maniatis, and E. F. Fritsch, Molecular Cloning: A Laboratory Manual, 2nd Ed. Cold Spring Harbor Laboratory Press, Cold Spring Harbor, NY., 1989.
Santos, R. and P. Flammang, Morphology and tenacity of the tube foot disc of three common European sea urchin species: a comparative study. Biofouling, 2006. 22 (3-4): p. 187-200.
Santos, R., et al., Adhesion of echinoderm tube feet to rough surfaces. J Exp Biol, 2005. 208 (Pt 13): p. 2555-67.
Santos, R., et al., First insights into the biochemistry of tube foot adhesive from the sea urchin Paracentrotus lividus (Echinoidea, Echinodermata). Mar Biotechnol (NY), 2009. 11 (6): p. 686-98.
Selsted, M. E., A. J. Ouellette. Mammalian defensins in the antimicrobial immune response. Nat Immunol. 2005 June; 6 (6): 551-7.
Schultz, M. P., Effects of coating roughness and biofouling on ship resistance and powering. Biofouling, 2007. 23 (5-6): p. 331-41.
Shai, Y., From innate immunity to de-novo designed antimicrobial peptides. Curr Pharm Des. 2002; 8 (9): 715-25.
Smith, A. M., The biochemistry and mechanics of gastropod adhesive gels. Biological adhesives, ed. C. J. e. Smith AM. 2006: Springer, Berlin. 167-182.
Snoek-van Beurden P. A., J. W. Von den Hoff. Zymographic techniques for the analysis of matrix metalloproteinases and their inhibitors. Biotechniques. 2005 January; 38 (1): 73-83.
Thomas, K. V. and S. Brooks, The environmental fate and effects of antifouling paint biocides. Biofouling. 26 (1): p. 73-88.
Townsin, R. L. and C. D. Anderson, Fouling control coatings using low surface energy, foul reliese technology. Woodhead Publishing Limited, Cambridge. 2009: p. 693-708.
Tsuru, D., (Microbial enzymes and their inhibitors). Yakugaku Zasshi, 1993. 113 (10): p. 683-97.
Vooturi, S. K. et al., Synthetic membrane-targeted antibiotics. Curr Med Chem. 2010; 17 (21): 2292-300.
Wang, Y., et al., The chemical characteristics of marine low-termperature alkaline protease Transactions of Oceanology and Limnology, 2004. 1: p. 8-15.
Watanabe, K., J. Sakai, and K. Hayano, Bacterial extracellular protease activities in field soils under different fertilizer managements. Can J Microbiol, 2003. 49 (5): p. 305-12.
Wilhelm, S., et al., Autotransporters with GDSL passenger domains: molecular physiology and biotechnological applications. Chembiochem, 2011. 12 (10): p. 1476-85.
Wu, J. W. and X. L. Chen, Extracellular metalloproteases from bacteria. Appl Microbiol Biotechnol, 2011. 92 (2): p. 253-62.
Yebra, D. M., S. Kiil, and K. Dam-Johansen, Antifouling technology-past, present and future steps towards efficient and environmentally friendly antifouling coatings. Progress in Organic Coatings, 2004. 50 (2): p. 75-104.
Zasloff, M. Innate immunity, antimicrobial peptides, and protection of the oral cavity. Lancet. 2002 Oct. 12; 360 (9340): 1116-7.

What is claimed is:

1. A method for preparing a bacterial supernatant comprising
i) culturing cells of Pseudomonas environmental strain PF-11; and
ii recovering the supernatant.

2. The method of claim 1, wherein the cells of Pseudomonas strain PF-11 are cultured under conditions at which the cells produce at least one extracellular protease, and the supernatant comprises the at least one extracellular protease.

3. The method of claim 1 or 2, wherein
(a) the supernatant is recovered when the number of cultured cells is increasing at an exponential rate;
(b) the supernatant is recovered after the number of cultured cells has ceased to increase at an exponential rate;
(c) the cells are cultured in a salts medium supplemented with glucose;
(d) the cells are cultured in M9 medium supplemented with glucose;
(e) the cells are cultured in medium which lacks ammonium and thyamine; or
(f) the cells are cultured at a temperature of about 28, 29, 30, 31, or 32° C.

4. The method of any one of claims 1-3, further comprising dividing the supernatant or modified supernatant into
(a) a fraction of components greater than 10 kilodaltons (kDa) in size; and
(b) a fraction of components less than 10 kDa in size.

5. The method of any one of claims 1-4, further comprising
(a) separating at least one extracellular protease from one or more components of the supernatant or a fraction thereof;
(b) reducing the salt concentration of the supernatant or a fraction thereof;
(c) reducing the water content of the supernatant a fraction thereof; or
(d) sterilizing the supernatant or a fraction thereof, so as to produce a modified supernatant or a fraction thereof.

6. The method of any one of claims 1-5, further comprising adding one or more acceptable carriers to the supernatant, modified supernatant, or fraction thereof.

7. A method for reducing the amount of a biofilm on a surface, comprising contacting the surface with
i) a supernatant, supernatant fraction, modified supernatant or modified supernatant fraction of Pseudomonas strain PF-11; or
ii a composition comprising a supernatant, supernatant fraction, modified supernatant or modified supernatant fraction of Pseudomonas strain PF-11, and one or more acceptable carriers.

8. The method of claim 7, wherein the biofilm
(a) is a fresh water biofilm;
(b) is a fresh water biofilm which is capable of growing in a pond, lake, or river environment;
(c) is a marine biofilm; or
(d) is capable of growing in a fresh or salt water aquarium.

9. A method for reducing adhesion of at least one organism to a surface, comprising contacting the surface with
i) a supernatant, supernatant fraction, modified supernatant or modified supernatant fraction of a Pseudomonas strain PF-11 culture; or
ii) a composition comprising a supernatant, supernatant fraction, modified supernatant or modified supernatant fraction of a Pseudomonas strain PF-11 culture, and one or more acceptable carriers.

10. The method of claim 9, wherein the at least one organism is an algae, a sea urchin, a barnacle, or a bryozoan zooid.

11. A method for reducing microfouling or macrofouling on a surface, comprising contacting the surface with
i) a supernatant, supernatant fraction, modified supernatant or modified supernatant fraction of a Pseudomonas strain PF-11 culture; or
ii) a composition comprising a supernatant, supernatant fraction, modified supernatant or modified supernatant fraction of a Pseudomonas strain PF-11 culture, and one or more acceptable carriers.

12. The method of and one of claims 7-11, wherein the surface is
(a) glass, fiberglass, wood, rubber, plastic, or metal;
(b) the surface of an aquarium, pool, buoy, dock, or hull of a ship or barge;
(c) a fishing net, or other net placed in water;
(d) a rope; or
(e) a wall or ceiling structure.

13. The method of any one of claims 7-12, wherein the composition comprising a supernatant, supernatant fraction, modified supernatant or modified supernatant fraction of a Pseudomonas strain PF-11 culture, and one or more acceptable carriers is a paint or transparent coating.

14. A method for killing or reducing the growth of a fungus, comprising contacting the fungi with
i) a supernatant, supernatant fraction, modified supernatant or modified supernatant fraction of a Pseudomonas strain PF-11 culture; or
ii) a composition comprising a supernatant, supernatant fraction, modified supernatant or modified supernatant fraction of a Pseudomonas strain PF-11 culture, and one or more acceptable carriers.

15. A method for killing or inhibiting the development of an insect, comprising contacting the insect with
i) a supernatant, supernatant fraction, modified supernatant or modified supernatant fraction of a Pseudomonas strain PF-11 culture; or
ii a composition comprising a supernatant, supernatant fraction, modified supernatant or modified supernatant fraction of a Pseudomonas strain PF-11 culture, and one or more acceptable carriers.

16. A method for killing or inhibiting the development of a marine copepod, comprising contacting the marine copepods with
i) a supernatant, supernatant fraction, modified supernatant or modified supernatant fraction of a Pseudomonas strain PF-11 culture; or
ii a composition comprising a supernatant, supernatant fraction, modified supernatant or modified supernatant fraction of a Pseudomonas strain PF-11 culture, and one or more acceptable carriers.

17. A method for killing or reducing the growth of a bacterial cell, comprising contacting the bacterial cell with
i) a supernatant, supernatant fraction, modified supernatant or modified supernatant fraction of a Pseudomonas strain PF-11 culture; or
ii a composition comprising a supernatant, supernatant fraction, modified supernatant or modified supernatant fraction of a Pseudomonas strain PF-11 culture, and one or more acceptable carriers.

18. The method of any one of claims 14-17, wherein the supernatant fraction or modified supernatant fraction comprises components greater than 10 kDa in size of a Pseudomonas strain PF-11 secretome.

19. The method of any one of claims 14-17, wherein the supernatant fraction or modified supernatant fraction comprises components less than 10 kDa in size of a Pseudomonas strain PF-11 secretome.

20. The method of any one of claims 17-19, wherein the bacterial cell is other than a Pseudomonas spp., Pseudomonas aeruginosa, or Pseudomonas cell.

21. The method of any one of claims 17-19, wherein the bacterial cell is a Staphylococcus spp., Staphylococcus aureus, or methicillin-resistant Staphylococcus aureus cell.

22. The method of any one of claims 17-19, wherein the bacterial cell is an Escherichia spp., Escherichia coli, or Escherichia coli O157 cell.

23. A substantially pure culture of Pseudomonas strain PF-11.

24. A substantially pure culture of Pseudomonas strain PF-11 cells, wherein the cells have been modified to comprise an exogenous resistance gene or an exogenous polynucleotide which encodes a reporter polypeptide operably linked to a promoter.

25. A substantially pure culture of Pseudomonas strain PF-11 cells, wherein the cells have been genetically modified to have increased susceptibility to an antibiotic compound compared to a corresponding cell of Pseudomonas strain PF-11.

26. The substantially pure culture of any one of claims 23-25, wherein less than about 40%; 35%; 30%; 25%; 20%; 15%; 10%; 5%; 2%; 1%; 0.5%; 0.25%; 0.1%; 0.01%; 0.001%; 0.0001%; or even less of the total number of viable microbial cells in the culture are viable cells other than the Pseudomonas strain PF-11 cells.

27. A bacterial culture that is enriched in Pseudomonas strain PF-11 cells.

28. The method of any one of claims 1-6 wherein the Pseudomonas strain PF-11 cell is a cell according to any one of claims 24-26.

29. The method of any one of claims 7-23, wherein the supernatant, supernatant fraction, modified supernatant or modified supernatant fraction is produced according to any one of claims 1-6.

30. The method of any one of claims 7-23 wherein the Pseudomonas strain PF-11 culture is a culture of one or more cells according to any one of claims 24-26.

31. A composition comprising
i) the cells of any one of claims 23-27, or a supernatant, modified supernatant, or fraction thereof, and
ii) one or more acceptable carriers.

32. An antifouling or antimicrobial composition comprising
i) the cells of any one of claims 23-27, or a supernatant, modified supernatant, or fraction thereof; or
ii) a composition comprising the cells of any one of claims 23-27, or a supernatant, modified supernatant or fraction thereof, and one or more acceptable carriers.

33. A method of identifying whether a bacteria is capable of producing one or more extracellular proteases capable of digesting a high molecular weight substrate comprising:
i) placing cells of the bacteria in a growth limiting medium supplemented with the high molecular weight substrate;
ii determining whether the cells grow in the growth limiting medium supplemented with the high molecular weight substrate; and
iii) identifying the bacteria as capable of producing one or more extracellular proteases capable of digesting the high molecular weight substrate if the cells are determined to grow in step ii), and identifying the bacteria as incapable of producing one or more extracellular proteases capable of digesting the high molecular weight substrate if the cells are determined to not grow in step ii).

34. The method of claim 33, wherein in step ii) the cells are determined to grow if the number of cells in the growth limiting medium supplemented with the high molecular weight substrate increases by at least 1, 5, 10, 100, 1000, or 10,000-fold over a period of at least 0.5, 1, 3, 4, 5, or 1-24 hours.

35. The method of claim 33 or 34, wherein
(a) the growth limiting medium is a salts medium;
(b) the growth limiting medium is a salts medium supplemented with glucose;
(c) the growth limiting medium is M9 medium supplemented with glucose;
(d) the growth medium lacks ammonium and thyamine;
(e) the growth limiting medium is maintained at a temperature of about 28, 29, 30, 31, or 32° C.;
(f) the growth limiting medium is a liquid; or
(g) the growth medium comprises agar.

36. The method of any one of claims 33-35, wherein high molecular weight substrate
(a) cannot pass through a cell wall or cell membrane of cells of the bacteria;
(b) must be degraded in order to be internalized and used for growth of the cell; or
(c) is gelatin, casein, hemoglobin, or bovine serum albumin (BSA).

37. The method of any one of claims 33-36 wherein the cells of the bacteria of step i) are obtained from a complete medium which is diluted into the growth limiting medium comprising the high molecular weight substrate.
